(19) United States
(12) Patent Application Publication
(10) Pub. No.: US 2011/0281911 A1
(43) Pub. Date: Nov. 17, 2011

(54) NOVEL FORMS OF EPERISONE

(75) Inventors: Isabel Kalofonos, Cambridge, MA (US); G. Patrick Stahly, West Lafayette, IN (US); William Martin-Doyle, Cambridge, MA (US); Dimitris Kalofonos, Cambridge, MA (US); Jeffrey S. Stults, West Lafayette, IN (US); Jason A. Hanko, West Lafayette, IN (US); Jeffrey P. Kiplinger, Boston, MA (US)

(73) Assignee: Bionevia Pharmaceuticals, Inc., Cambridge, MA (US)

(21) Appl. No.: 13/057,542

(22) PCT Filed: Aug. 3, 2009
(86) PCT No.: PCT/US09/52575

§ 371 Date: Aug. 3, 2011

Related U.S. Application Data
(60) Provisional application No. 61/086,020, filed on Aug. 4, 2008.

(51) Int. Cl. A61K 31/4453; A61P 21/00; A61P 25/00; A61P 29/00; A61P 25/14; C07D 295/108; A61P 21/02

(52) U.S. Cl. 514/317; 546/237

(57) ABSTRACT

The invention relates to substantially enantiopure crystalline salt forms of (2RS)-I-(4-Ethylphenyl)-2-methyl-3-piperidin-1-ylpropan-1-one. The preparation and characterization of the substantially enantiopure crystalline salt forms according to various embodiments of the invention is described. The invention also relates to pharmaceutical compositions containing the substantially enantiopure crystalline salt forms, which are useful to treat and/or prevent various conditions such as pathological muscle contracture, myotome conditions, and spastic paralysis or spasticity caused by various neurologic conditions, and are also useful for the treatment and/or prevention of various types of pain and pathological muscle tension.

### CROSS-REFERENCE TO RELATED APPLICATIONS

This application claims priority under 35 U.S.C. §119 to U.S. Provisional Application 61/086,020, filed Aug. 4, 2008, which is incorporated herein by reference.

### TECHNICAL FIELD

The invention relates to substantially enantiopure novel crystalline forms of (2RS)-1-(4-Ethylphenyl)-2-methyl-3-piperidin-1-ylpropan-1-one, processes for making those novel crystalline forms, pharmaceutical compositions comprising those novel crystalline forms, and methods of treating and/or preventing various conditions by administering those novel crystalline forms.

### BACKGROUND

The compound (2RS)-1-(4-Ethylphenyl)-2-methyl-3-piperidin-1-ylpropan-1-one (shown below), referred to herein by its common name “eperisone,” is a known active pharmaceutical ingredient (API) having beneficial therapeutic activity, for example as a muscle relaxant and spasmolytic, and is useful in treating various conditions including pathological muscle contracture resulting from a variety of underlying musculoskeletal and neurologic conditions:

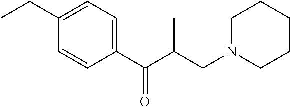

Racemic eperisone hydrochloride has a positive indication for the improvement of myotonic conditions caused by neck-shoulder-arm syndrome, scapulohumeral periarthritis, and low back pain, and for spastic paralysis or spasticity caused by various neurologic conditions, and is also useful for the treatment of various types of pain and pathological muscle tension. The preparation and pharmacologic activity of racemic eperisone hydrochloride is described for example in U.S. Pat. No. 3,995,047. Therapeutic activity in various conditions has been demonstrated in the clinical literature, for example in Bose K., Methods Find Exp Clin Pharmacol (1999) 21:209-13; Hanai K. et al., Jpn J Clin Exp Med (1983) 60:2049-2053; Hirohata K. et al., J New Remed Clin (1988) 37:200; Iwasaki T. et al., Nippon Ganka Gakkai Zasshi (1987) 91:740-6; Iwase S. et al., Funct Neurol. (1992) 7:459-70; Kobayashi Y. et al., Dig Dis Sci. (1992) 37:1145-6; Kuroiwa Y. et al., Jpn J Clin Exp Med (1980) 57:4033-4038; Kuroiwa Y. et al., Clin. Eval. (1981) 9:391-419; Mano T. et al., No To Shinkei (1981) 33:237-41; Mizuno K. et al., Prog Med (1991) 11:99-112; Murayama K. et al., Hinyokika Kiyo (1984) 30:403-8; Nakahara S. et al., Prog Med (1986) 6:11; Takayasu et al, Oncology (1989) 46(1): 58-60; and Nisijima K. et al., Acta Psychiatr Scand (1998) 98:341-3; U.S. Pat. No. 5,002,958; WO2004/089352; and U.S. Patent Application No. 20060004050.

The separation of racemic eperisone hydrochloride into its optical isomers (+)-eperisone and (−)-eperisone, is described in U.S. Pat. No. 6,143,180, Matsunaga H. et al., Electrophoresis (2003) 24:2442-2447; Matsunaga H. et al., Electrophoresis (2001) 22:3251-3256; Haginaka J. et al., Journal of Chromatography A (1997) 782:281-288; Tsukumoto T. et al., Journal of Chromatography A (1999) 864:163-171; Tanaka Y. et al., Journal of High Resolution Chromatography (1996) 19:421; Ishihama Y. et al., Journal of Chromatography A (1994) 666:193-201; and Tanaka Y. et al., Chromatographia (1997) 44:119.

Although therapeutic efficacy is a primary concern for a therapeutic agent, such as eperisone, the salt and solid state form (e.g. crystalline or amorphous forms) of a drug candidate can be important to its pharmacological properties and to its development as a viable API. For example, each salt or each crystalline form of a drug candidate can have different solid state (physical and chemical) properties. The differences in physical properties exhibited by a particular solid form of an API, such as a cocrystal, salt, or polymorph of the original compound, can affect pharmaceutical parameters of the API. For example, storage stability, compressibility and density, all of which can be important in formulation and product manufacturing, and solubility and dissolution rates, which may be important factors in determining bioavailability, may be affected. Because these physical properties are often influenced by the solid state form of the API, they can significantly impact a number of factors, including the selection of a compound as an API, the ultimate pharmaceutical dosage form, the optimization of manufacturing processes, and absorption in the body. Moreover, finding the most adequate form for further drug development can reduce the time and the cost of that development.

Obtaining pure crystalline forms, then, is extremely useful in drug development. It may permit better characterization of the drug candidate's chemical and physical properties. For example, crystalline forms often have better chemical and physical properties than amorphous forms. As a further example, a crystalline form may possess more favorable pharmacology than an amorphous form, or may be easier to process. It may also have better storage stability.

One such physical property which can affect processability is the flowability of the solid, before and after milling. Flowability affects the ease with which the material is handled during processing into a pharmaceutical composition. When particles of the powdered compound do not flow past each other easily, a formulation specialist must take that fact into account in developing a tablet or capsule formulation, which may necessitate the use of additional components such as glidants, including colloidal silicon dioxide, talc, starch, or tribasic calcium phosphate.

Another solid state property of a pharmaceutical compound that may be important is its dissolution rate in aqueous fluid. The rate of dissolution of an active ingredient in a patient's stomach fluid may have therapeutic consequences since it can impact the rate at which an orally administered active ingredient may reach the patient's bloodstream.

Another solid state property of a pharmaceutical compound that may be important is its thermal behavior, including its melting point. The melting point of the solid form of a drug is optionally high enough to avoid melting or plastic deformation during standard processing operations, as well as concretion of the drug by plastic deformation on storage (See, e.g., Gould, P. L. Int. J. Pharmaceutics 1986 33 201-217). It may be desirable in some cases for a solid form to melt above about 100° C. For example, melting point categories used by one pharmaceutical company are, in order of preference, + (mp>120° C.), 0 (mp 80-120° C.), and − (mp<80° C.) (Balbach, S.; Korn, C. Int. J. Pharmaceutics 2004 275 1-12).

Active drug molecules may be made into pharmaceutically acceptable salts for therapeutic administration to the patient. Crystalline salts of a drug may offer advantages over the free form of the compound, such as improved solubility, stability, processing improvements, etc., and different crystalline salt forms may offer greater or lesser advantages over one another. However, crystalline salt formation is not predictable, and in fact is not always possible. Moreover, there is no way to predict the properties of a particular crystalline salt of a compound until it is formed. As such, finding the right conditions to obtain a particular crystalline salt form of a compound, with pharmaceutically acceptable properties, can take significant time and effort.

A crystalline form of a compound, a crystalline salt of the compound, or a cocrystal containing the compound or its salt form generally possesses distinct crystallographic and spectroscopic properties when compared to other crystalline forms having the same chemical composition. Crystallographic and spectroscopic properties of a particular form may be measured by XRPD, single crystal X-ray crystallography, solid state NMR spectroscopy, e.g. ¹³C CP/MAS NMR, or Raman spectroscopy, among other techniques. A particular crystalline form of a compound, of its salt, or of a cocrystal, often also exhibits distinct thermal behavior. Thermal behavior can be measured in the laboratory by such techniques as, for example, capillary melting point, TGA, and DSC.

Many organic compounds can exist as optically active forms, i.e., they have the ability to rotate the plane of plane-polarized light. In describing an optically active compound the prefixes R- and S-, and D- and L-, are used to denote the absolute configuration of the molecule about its chiral center(s). The prefixes d- and l-, or (+)- or (−)-, designate the sign of rotation of plane-polarized light by the compound, with l- or (−)- meaning that the compound is levorotatory. In contrast, a compound prefixed with d- or (+)- is dextrorotatory. There is no correlation between nomenclature for the absolute stereochemistry and for the rotation of light by an enantiomer. By way of example, d-lactic acid is the same as (−)-lactic acid, and l-lactic acid is the same as (+)-lactic acid. For a given chemical structure, each of a pair of enantiomers is identical except that they are non-superimposable mirror images of one another. In general, enantiomers have identical properties in a symmetrical environment, although their properties may differ in an unsymmetrical environment. A mixture of enantiomers is often called an enantiomeric, or racemic, mixture, or a racemate.

Stereochemical purity may be particularly important in the pharmaceutical field, where many of the most often prescribed drugs exhibit chirality. For example, the L-enantiomer of the beta-adrenergic blocking agent, propranolol, is known to be 100 times more potent than its D-enantiomer. Additionally, optical purity may be important in the pharmaceutical drug field because certain isomers have been found to impart a deleterious effect, rather than an advantageous or inert effect. For example, it is believed that the D-enantiomer of thalidomide is a safe and effective sedative when prescribed for the control of morning sickness during pregnancy, whereas its corresponding L-enantiomer is believed to be a potent teratogen.

Currently, eperisone is available only as a racemic mixture of enantiomers, (+)- and (−)- in a 1:1 ratio, and reference herein to the generic name “eperisone” refers to this enantiomeric, or racemic, mixture. Racemic eperisone hydrochloride is commercially sold under the trade name MYONAL. Administration of racemic eperisone, however, can result in certain undesirable side effects such as, for example, insomnia, headache, nausea and vomiting, anorexia, abdominal pain, diarrhea, constipation, urinary retention, and/or incontinence, at least some of which may be avoided by the use of a pure enantiomer of the compound. Substantially enantiopure crystalline eperisone hydrochloride has not heretofore been reported in the literature.

As used herein, the statement “(+)-enantiomer substantially free of the (−)-enantiomer” is meant to describe, for example, a compound that comprises about 80% or more by weight of the (+)-enantiomer, and contains about 20% or less by weight of the (−)-enantiomer, such as greater than about 90% by weight, greater than about 95% by weight, and greater than about 99% by weight, based on the total weight of the active ingredient. Likewise, the statement “(−)-enantiomer substantially free of the (+)-enantiomer” is meant to describe, for example, a compound that comprises about 80% or more by weight of the (−)-enantiomer, and contains about 20% or less by weight of the (+)-enantiomer, such as greater than about 90% or more by weight of the (−)-enantiomer, and contains less than about 10% by weight of the (+)-enantiomer, greater than about 95% by weight, and greater than about 99% by weight of the (−)-enantiomer, based on the total weight of the active ingredient. The term “enantiopure” or “substantially enantiopure” as used herein is meant to include, for example, compounds that comprise about 80% or more, such as about 90% or more, about 95% or more, or about 99% or more, of one enantiomer of the referenced compound.

Although there are many procedures known in the art for separating enantiomers or synthesizing enantiopure compounds, not all procedures work for all compounds. Thus, much time and effort is often spent devising a procedure that is effective for separating or synthesizing the desired enantiomer of many compounds.

In the following description, various aspects and embodiments of the invention will become evident. In its broadest sense, the invention could be practiced without having one or more features of these aspects and embodiments. Further, these aspects and embodiments are exemplary. Additional objects and advantages of the invention will be set forth in part in the description which follows, and in part will be obvious from the description, or may be learned by practicing of the invention. The objects and advantages of the invention will be realized and attained by means of the elements and combinations particularly pointed out in the appended claims.

### SUMMARY

In accordance with various embodiments of the invention and after extensive experimentation, the inventors have discovered substantially enantiopure novel crystalline salt forms of eperisone, including crystalline forms of (+)-eperisone hydrochloride, (−)-eperisone hydrochloride, (+)-eperisone mesylate, (−)-eperisone mesylate, (+)-eperisone maleate, and (−)-eperisone maleate.

The invention in various embodiments also relates to processes of preparing those substantially enantiopure crystalline salts of eperisone, pharmaceutical compositions containing them, and their use in the treatment and/or prevention of various conditions including, for example, myotonic conditions, pain, and pathological muscle tension, as well as improving blood flow.

As used herein, the term “XRPD” refers to x-ray powder diffraction. The XRPD data disclosed herein were obtained using an Inel XRG-3000 diffractometer equipped with a CPS (Curved Position Sensitive) detector with a 2θ range of 120°. Real time data were collected using Cu-Kα radiation at a resolution of 0.03°2θ. The tube voltage and amperage were set to 40 kV and 30 mA, respectively. The monochromator slit was set at 1-5 mm by 160 μm. Samples were prepared for analysis by packing them into thin-walled glass capillaries. Each capillary was mounted onto a goniometer head that is motorized to permit spinning of the capillary during data acquisition. Instrument calibration was performed using a silicon reference standard.

As used herein, the term “DSC” refers to differential scanning calorimetry. DSC data disclosed herein were obtained using a TA Instruments differential scanning calorimeter 2920 or Q2000. The sample was placed into an aluminum DSC pan, and the weight accurately recorded. The pan was crimped and the contents heated under nitrogen under the conditions given in the figures. Indium metal was used as the calibration standard.

As used herein, the term “¹H-NMR” refers to proton nuclear magnetic resonance spectroscopy. Solution ¹H NMR data disclosed herein were acquired on a Varian ^{UNITY}INOVA-400 spectrometer (¹H Larmor Frequency=399.8 MHz). The specific parameters of each spectrum are provided on the attached figures.

As used herein, the term “TGA” refers to thermogravimetric analysis. TGA data disclosed herein were obtained using a TA Instruments 2950 thermogravimetric analyzer. Each sample was placed in an aluminum sample pan and inserted into the TG furnace. The heating conditions are shown in the figures. Nickel and Alumel™ were used as the calibration standards. Reported temperatures are at the transition maxima.

As described herein, optical microscopy was performed using a Leica MZ12.5 stereomicroscope. Samples were viewed in situ or on a glass slide (covered in Paratone-N oil) through crossed polarizers and a first order red compensator using various objectives ranging from 0.8-10×.

As used herein, “Raman” refers to Raman spectroscopy. Raman was performed in one of two ways: (1) dispersive Raman spectra were acquired on a Renishaw Mk1 Ramascope model 1000 equipped with a Leica DM LM microscope. The samples were prepared for analysis by placing particles onto a gold mirror. The instrument was calibrated with a silicon wafer standard and a neon emission lamp, and (2) FT-spectra were acquired on a Raman accessory module interfaced to a Magna 860 or 960® Fourier transform infrared (FT-IR) spectrophotometer (Thermo Nicolet), which use an excitation wavelength of 1064 nm and an indium gallium arsenide (InGaAs) detector. The samples were prepared for analysis by placing the material in a glass tube and positioning the tube in a gold-coated tube holder in the accessory. A specified number of sample scans were collected using Happ-Genzel apodization. Specific parameters are printed on each spectrum in the data section. Wavelength calibration was performed using sulfur and cyclohexane. The specific parameters of each spectrum are provided on the attached figures.

As used herein. “HPLC” means high performance liquid chromatography. Samples were weighed and dissolved in HPLC-grade water at the concentration of either ˜0.2 mg/mL or ˜0.1 mg/mL. The filtrates were diluted to ˜0.1 mg/mL with HPLC-grade water. All prepared samples were wrapped with aluminum foil to avoid light and ˜1.5 mL of each were transferred to amber auto-sampler vials for the analysis. All HPLC analyses were performed using an Agilent 1100 series liquid chromatograph equipped with a diode array detector, degasser, quaternary pump, and an autosampler. The chromatographic column was a 4.6×150 mm Chiralpak AD-RH column with 5.0 μm packing (Chiral Technologies Inc.). The column temperature was set to 30° C. and the detector wavelength was 260 nm, with a bandwidth of 8 nm and a reference wavelength of 360 nm. The injection volume was 10 μL. Mobile phase A was pH 9.0 borate buffer. Mobile phase B was 100% acetonitrile. The method used an isocratic run at 40:60 borate buffer to acetonitrile for 30 minutes. The flow rate was set at 0.5 mL/minute

As used herein with respect to the various analytical techniques described herein and data generated therefrom, the term “substantially” the same as or similar to is meant to convey that a particular set of analytical data is, within acceptable scientific limits, sufficiently similar to that disclosed herein such that one of skill in the art would appreciate that the crystal salt form of the compound is the same as that of the present invention. One of skill in the art would appreciate that certain analytical techniques, such as, for example, XRPD, ¹H-NMR, DSC, TGA, and Raman, will not produce exactly the same results every time due to, for example, instrumental variation, sample preparation, scientific error, etc. By way of example only. XRPD results (i.e. peak locations, intensities, and/or presence) may vary slightly from sample to sample, despite the fact that the samples are, within accepted scientific principles, the same form, and this may be due to, for example, preferred orientation or varying solvent or water content. It is well within the ability of those skilled in the art, looking at the data as a whole, to appreciate whether such differences indicate a different form, and thus determine whether analytical data being compared to those disclosed herein are substantially similar. In this regard, and as is commonly practiced within the scientific community, it is not intended that the exemplary analytical data of the novel salts of eperisone disclosed herein be met literally in order to determine whether comparative data represent the same form as those disclosed and claimed herein, such as, for example, whether each and every peak of an exemplary XRPD pattern of a novel crystalline salt of eperisone disclosed herein is present in the comparative data, in the same location, and/or of the same intensity. Rather, as discussed above, it is intended that those of skill in the art, using accepted scientific principles, will make a determination based on the data as a whole regarding whether comparative analytical data represent the same or a different form of any of the novel crystalline salts of eperisone disclosed herein.

It is to be understood that both the foregoing general description and the following detailed description are exemplary and explanatory only and are not restrictive of the invention, as claimed.

### BRIEF DESCRIPTION OF THE FIGURES

FIG. 1A is an XRPD pattern of a substantially enantiopure crystalline hydrochloride salt of eperisone, according to one embodiment of the invention;

FIG. 1B is a comparison of two XRPD patterns of substantially enantiopure crystalline hydrochloride salts of eperisone, wherein one sample is (+)-eperisone hydrochloride and the other is (−)-eperisone hydrochloride;

FIG. 2A is an XRPD pattern of a substantially enantiopure crystalline mesylate salt of eperisone, according to one embodiment of the invention;

FIG. 2B is a comparison of two XRPD patterns of substantially enantiopure crystalline mesylate salts of eperisone, wherein one sample is (+)-eperisone mesylate and the other sample is (−)-eperisone mesylate;

FIG. 3A is an XRPD pattern of a substantially enantiopure crystalline maleate salt of eperisone, according to one embodiment of the invention;

FIG. 3B is a comparison of two XRPD patterns of substantially enantiopure crystalline maleate salts of eperisone, wherein one sample is (+)-eperisone maleate and the other sample is (−)-eperisone maleate;

FIG. 4 is a DSC thermogram of a substantially enantiopure crystalline hydrochloride salt of eperisone, according to one embodiment of the invention;

FIG. 5 is a DSC thermogram of a substantially enantiopure crystalline mesylate salt of eperisone, according to one embodiment of the invention;

FIG. 6 is a DSC thermogram of a substantially enantiopure crystalline maleate salt of eperisone, according to one embodiment of the invention;

FIG. 7A is a full ¹H-NMR spectrum of a substantially enantiopure crystalline hydrochloride salt of eperisone, according to one embodiment of the invention;

FIG. 7B is an ¹H-NMR spectrum from 8.8 to 7.3 ppm of a substantially enantiopure crystalline hydrochloride salt of eperisone, according to one embodiment of the invention;

FIG. 7C is an ¹H-NMR spectrum from 5.5 to 4.5 ppm of a substantially enantiopure crystalline hydrochloride salt of eperisone, according to one embodiment of the invention;

FIG. 7D is an ¹H-NMR spectrum from 4.3 to 3.3 ppm of a substantially enantiopure crystalline hydrochloride salt of eperisone, according to one embodiment of the invention;

FIG. 7E is an ¹H-NMR spectrum from 3.3 to 2.6 ppm of a substantially enantiopure crystalline hydrochloride salt of eperisone, according to one embodiment of the invention;

FIG. 7F is ¹H-NMR spectrum from 2.48 to 2.20 ppm of a substantially enantiopure crystalline hydrochloride salt of eperisone, according to one embodiment of the invention;

FIG. 7G is an ¹H-NMR spectrum from 2.1 to 0.7 ppm of a substantially enantiopure crystalline hydrochloride salt of eperisone, according to one embodiment of the invention;

FIG. 8A is a full ¹H-NMR spectrum of a substantially enantiopure crystalline mesylate salt of eperisone, according to one embodiment of the invention;

FIG. 8B is an ¹H-NMR spectrum from 9.2 to 7.2 ppm of a substantially enantiopure crystalline mesylate salt of eperisone, according to one embodiment of the invention;

FIG. 8C is an ¹H-NMR spectrum 4.4 to 2.6 ppm of a substantially enantiopure crystalline mesylate salt of eperisone, according to one embodiment of the invention;

FIG. 8D is an ¹H-NMR spectrum from 2.6 to 1.0 ppm of a substantially enantiopure crystalline mesylate salt of eperisone, according to one embodiment of the invention;

FIG. 9A is a full ¹H-NMR spectrum of a substantially enantiopure crystalline maleate salt of eperisone, according to one embodiment of the invention;

FIG. 9B is an ¹H-NMR spectrum from 10.0 to 6.0 ppm of a substantially enantiopure crystalline maleate salt of eperisone, according to one embodiment of the invention;

FIG. 9C is an ¹H-NMR spectrum from 4.5 to 1.0 ppm of a substantially enantiopure crystalline maleate salt of eperisone, according to one embodiment of the invention;

FIG. 10 is TGA profile of a substantially enantiopure crystalline hydrochloride salt of eperisone, according to one embodiment of the invention;

FIG. 11 is TGA profile of a substantially enantiopure crystalline mesylate salt of eperisone, according to one embodiment of the invention;

FIG. 12 is TGA profile of a substantially enantiopure crystalline maleate salt of eperisone, according to one embodiment of the invention;

FIG. 13A is an FT-Raman spectrum of a substantially enantiopure crystalline hydrochloride salt of eperisone, according to one embodiment of the invention;

FIG. 13B is a dispersive Raman spectrum of a substantially enantiopure crystalline hydrochloride salt of eperisone, according to one embodiment of the invention;

FIG. 14A is an FT-Raman spectrum of a substantially enantiopure crystalline mesylate salt of eperisone, according to one embodiment of the invention;

FIG. 14B is a dispersive Raman spectrum of a substantially enantiopure crystalline mesylate salt of eperisone, according to one embodiment of the invention; and

FIG. 15 is an FT-Raman spectrum of a substantially enantiopure crystalline maleate salt of eperisone, according to one embodiment of the invention.

### DESCRIPTION OF EXEMPLARY EMBODIMENTS

The invention relates to substantially enantiopure novel crystalline salt forms of eperisone. Specifically, the novel crystalline salts which have been discovered include crystalline forms of (+)-eperisone hydrochloride, (−)-eperisone hydrochloride, (+)-eperisone mesylate, (−)-eperisone mesylate, (+)-eperisone maleate, and (−)-eperisone maleate. In one exemplary embodiment of the invention, these novel crystalline salts of eperisone may be in substantially enantiopure form. Exemplary methods of preparation of the novel crystalline salt forms of eperisone, such as the substantially enantiopure novel crystalline salt forms of eperisone, according to various embodiments of the invention are described below in the examples.

In addition, methods for increasing the enantiomeric purity of crystalline eperisone mesylate are also disclosed.

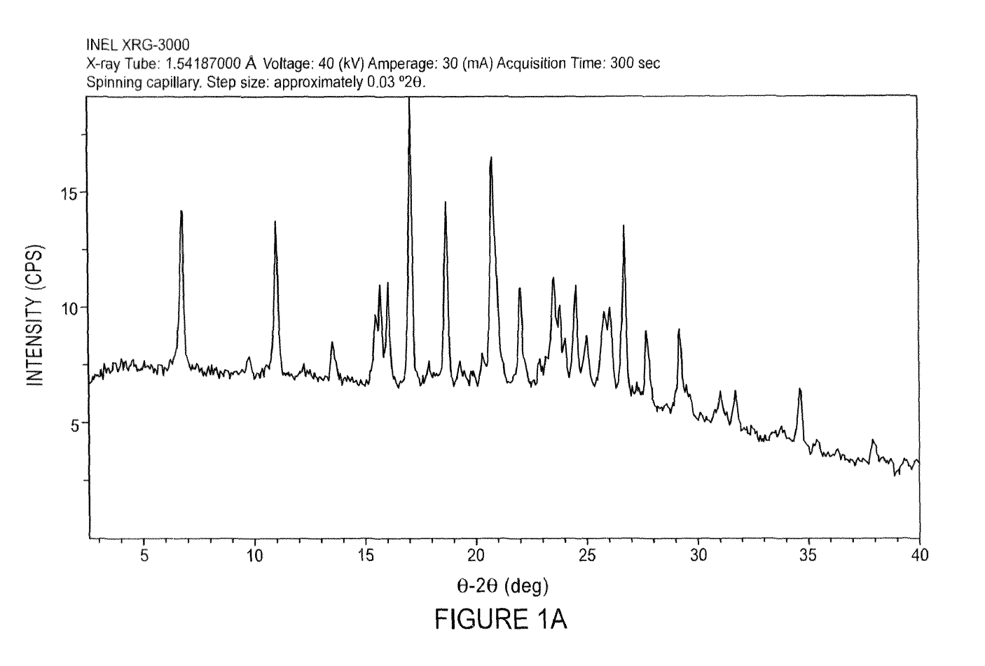

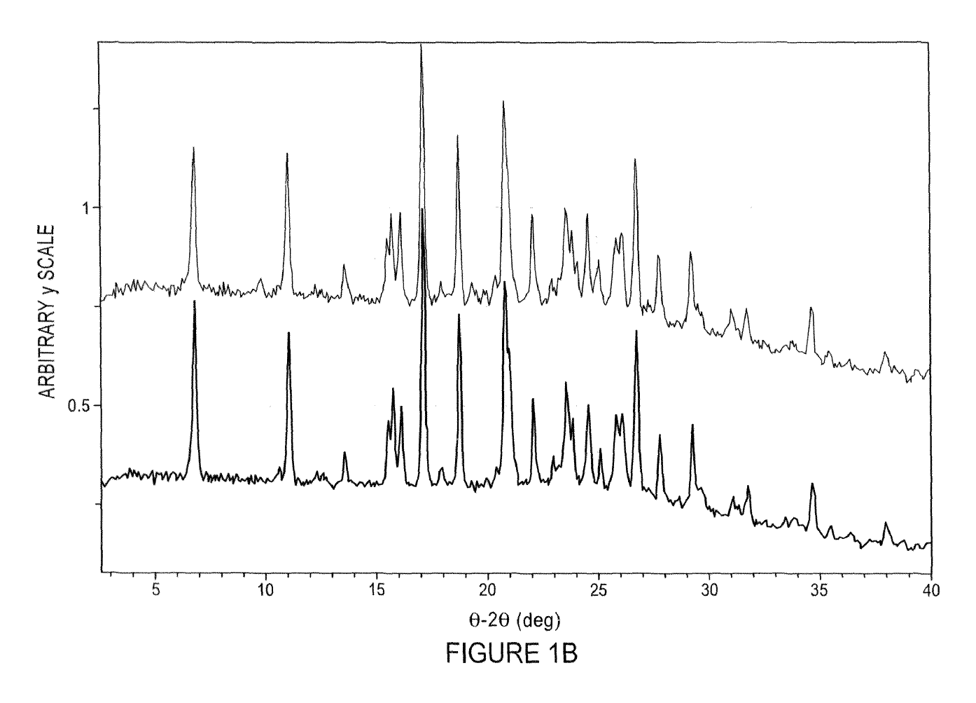

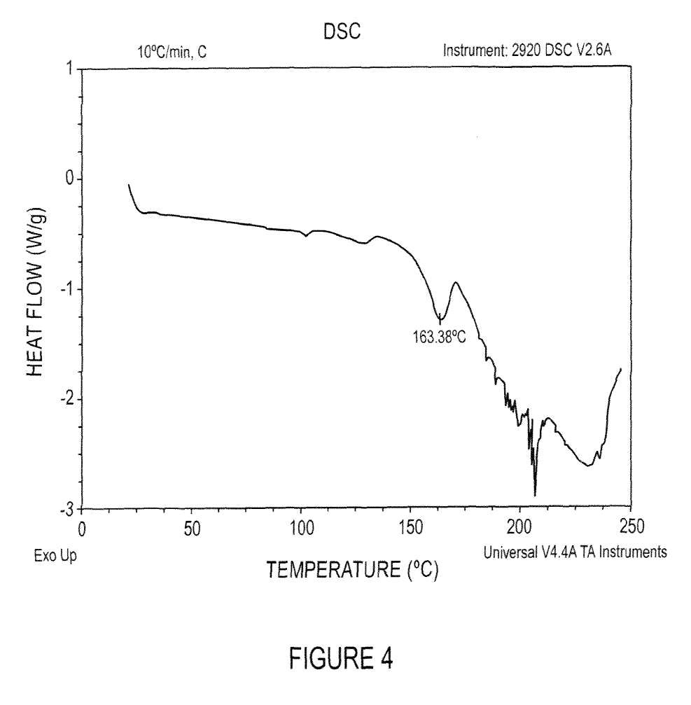

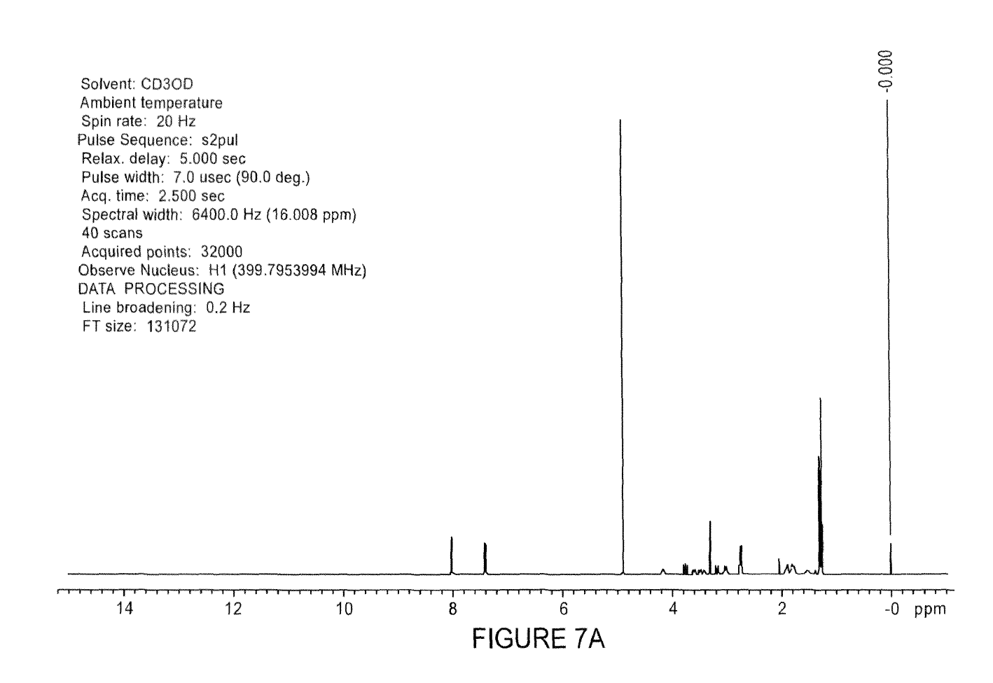

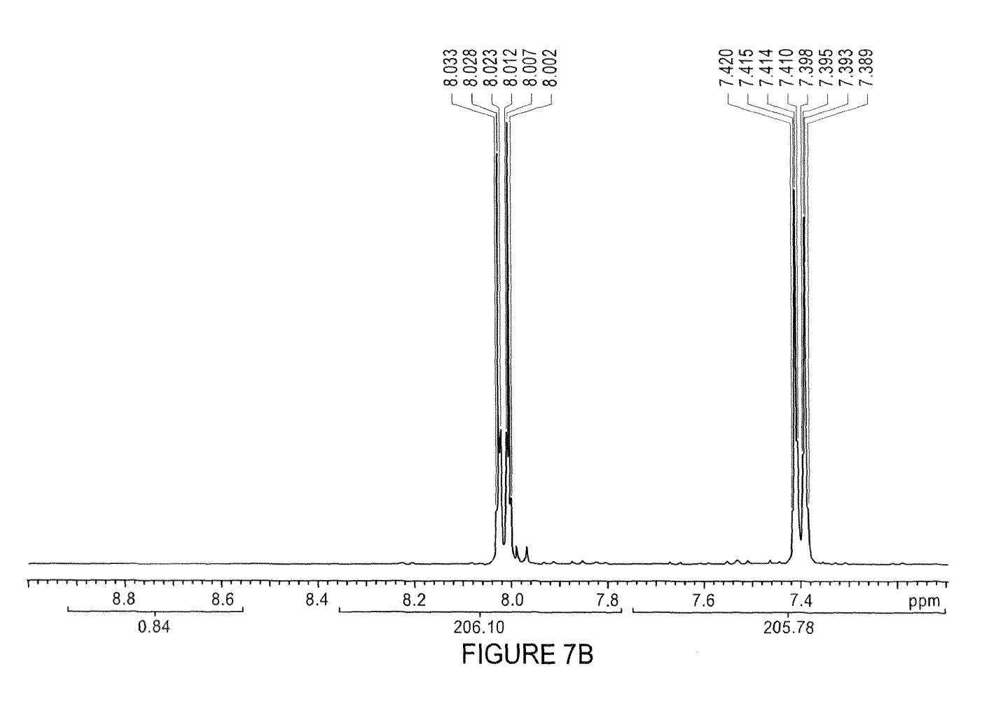

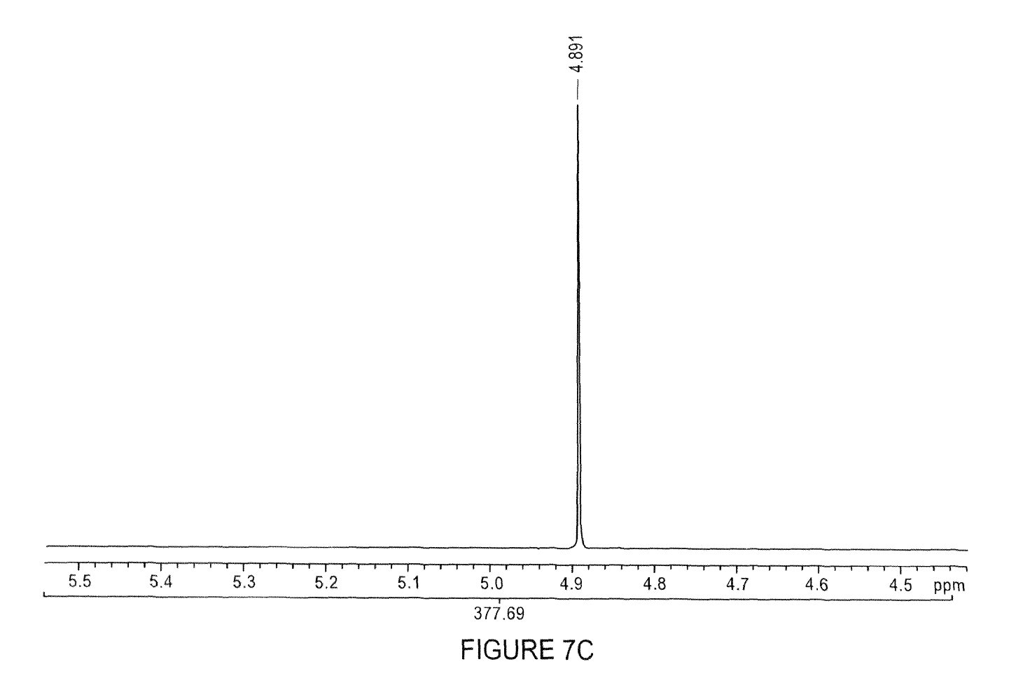

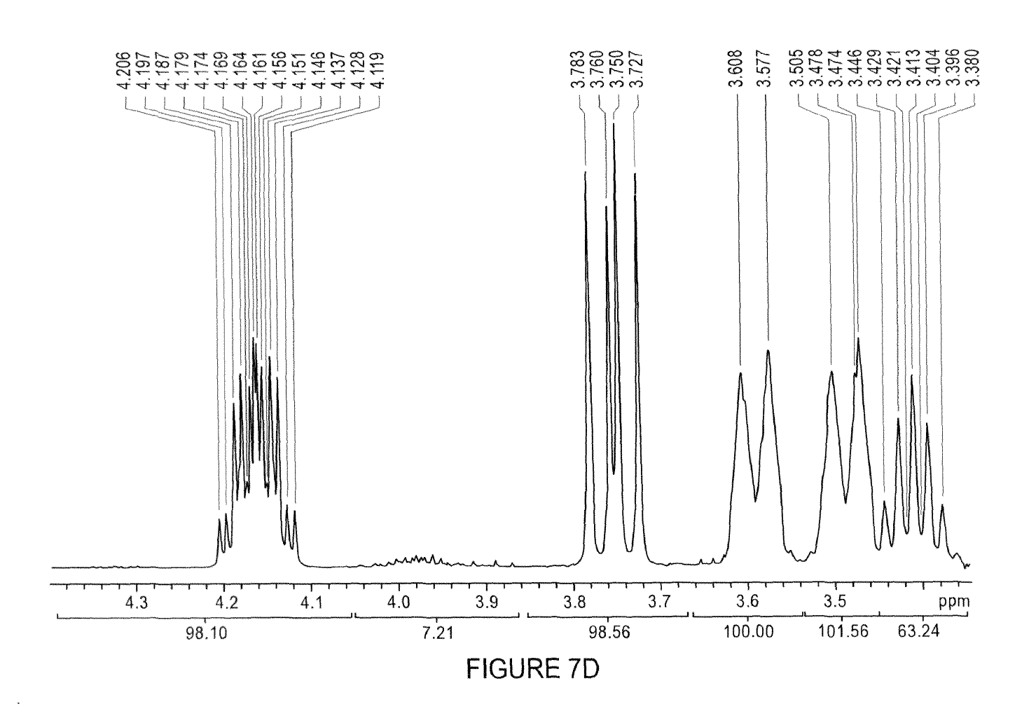

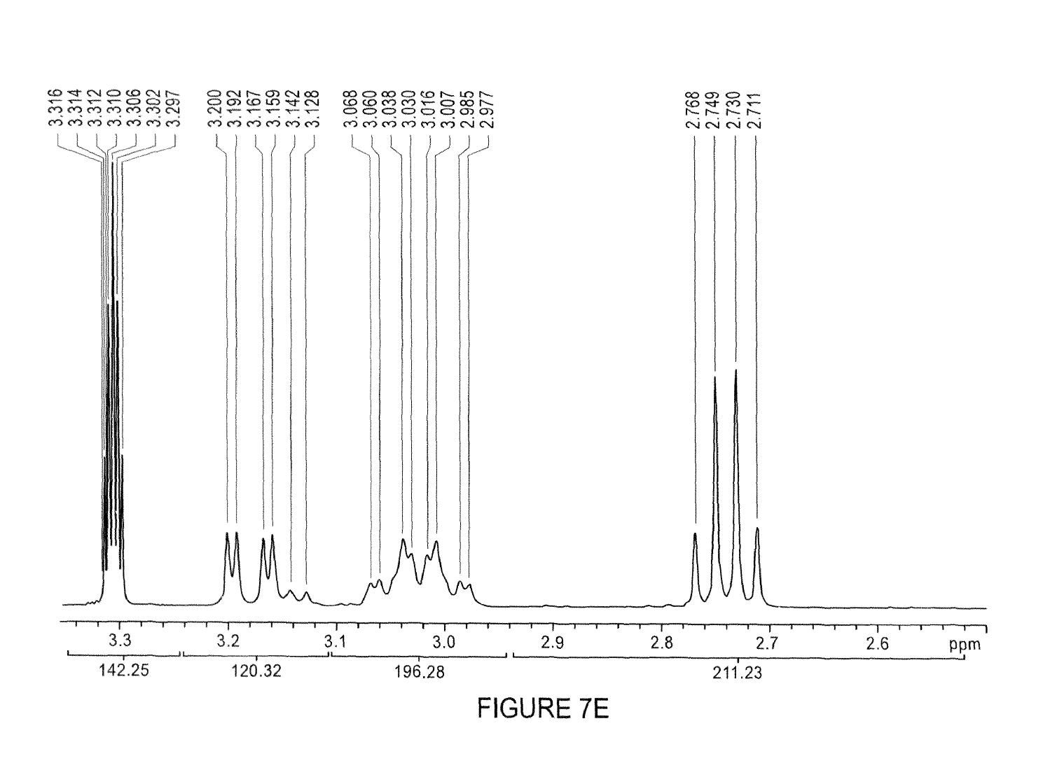

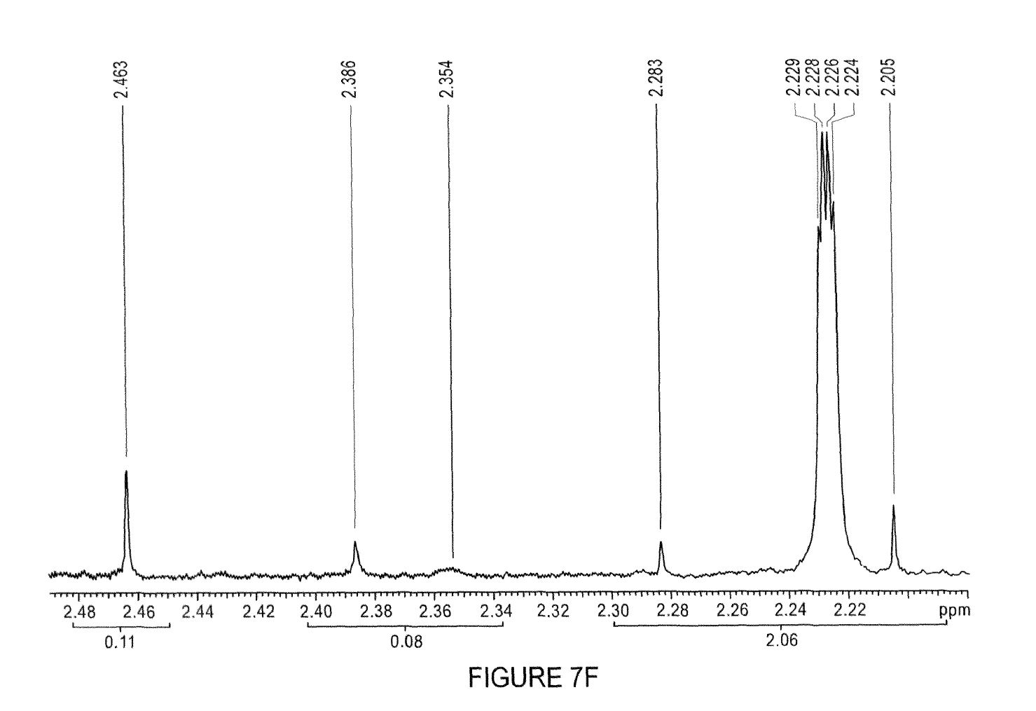

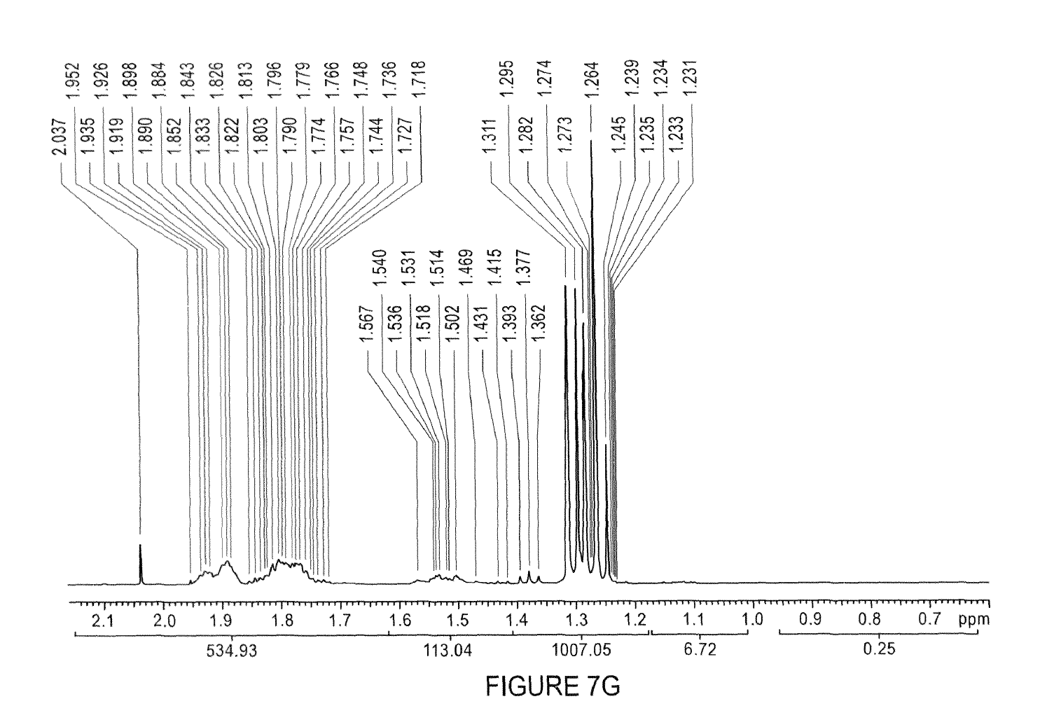

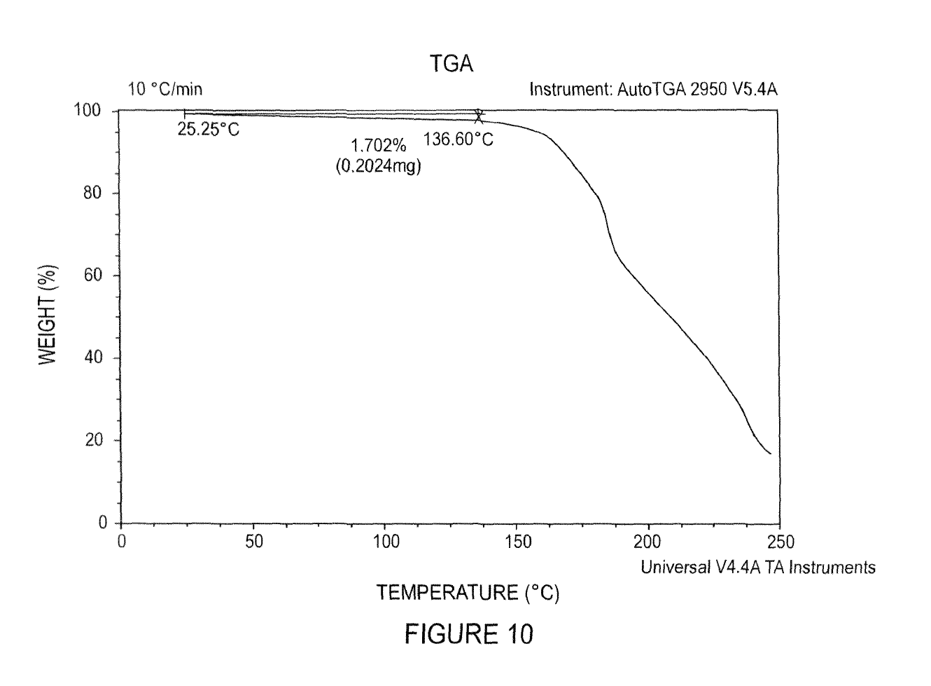

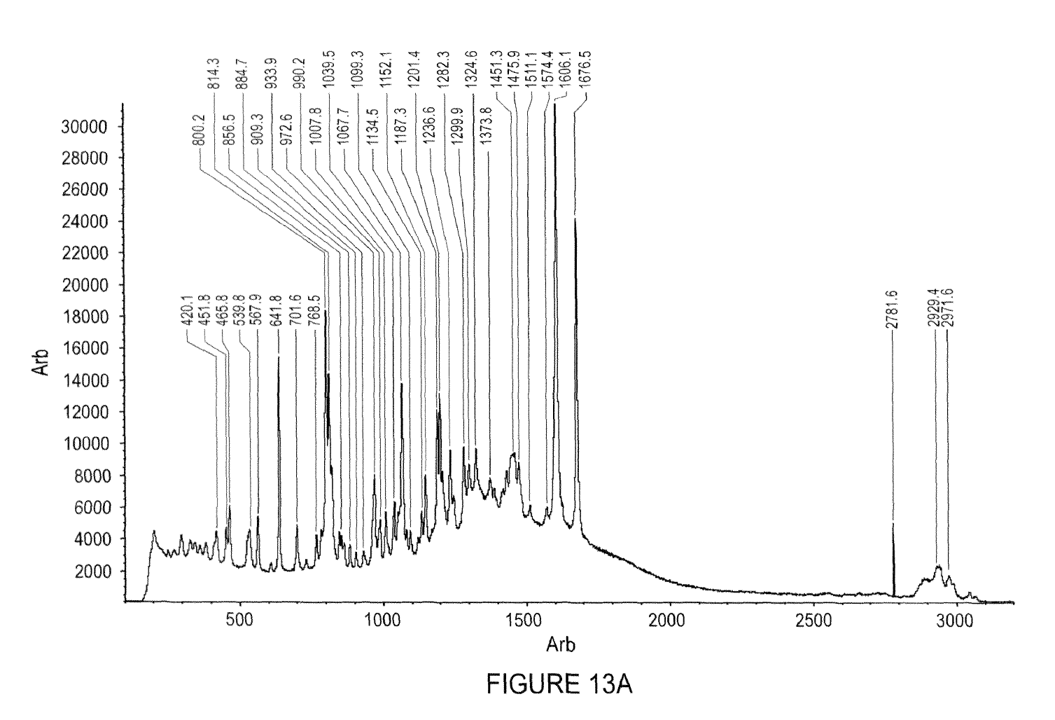

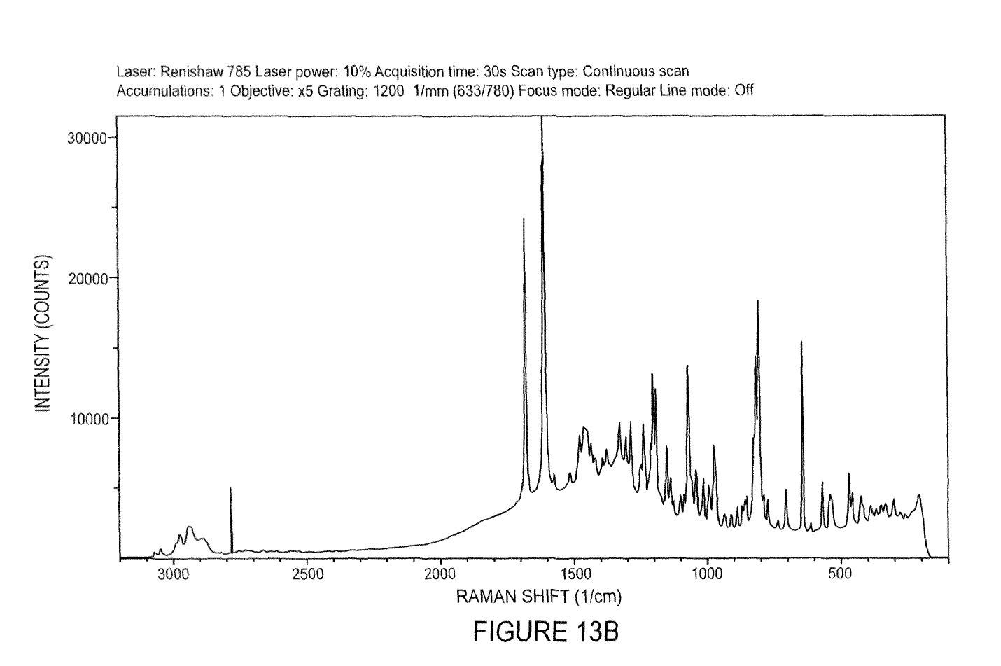

Substantially enantiopure crystalline eperisone hydrochloride is characterized by an XRPD pattern substantially as shown in FIG. 1A, a DSC thermogram substantially as shown in FIG. 4, an ¹H-NMR spectrum substantially as shown in FIGS. 7A-7G, a TGA profile substantially as shown in FIG. 10, and Raman spectra substantially as shown in FIGS. 13A and 13B. FIG. 1B demonstrates that (+)-eperisone hydrochloride and (−)-eperisone hydrochloride have substantially similar XRPD patterns. An exemplary listing of representative XRPD peaks of a substantially enantiopure crystalline eperisone hydrochloride salt according to an embodiment of the invention can be found in Table 1. An exemplary listing of representative NMR data, obtained in CD₃OD, can be found in Table 2.

TABLE 1

| Degrees 2θ | | d spacing Å | | Intensity % (I/Io) |
|---|---|---|---|---|
| 6.8 | ±0.2 | 12.98 | ±0.39 | 57 |
| 9.7 | ±0.2 | 9.11 | ±0.19 | 8 |
| 11.0 | ±0.2 | 8.03 | ±0.15 | 55 |
| 13.5 | ±0.2 | 6.55 | ±0.10 | 15 |
| 15.5 | ±0.2 | 5.71 | ±0.07 | 25 |
| 15.7 | ±0.2 | 5.64 | ±0.07 | 35 |
| 16.1 | ±0.2 | 5.50 | ±0.07 | 36 |
| 17.1 | ±0.2 | 5.18 | ±0.06 | 100 |
| 17.9 | ±0.2 | 4.95 | ±0.06 | 9 |
| 18.7 | ±0.2 | 4.74 | ±0.05 | 64 |
| 19.3 | ±0.2 | 4.59 | ±0.05 | 9 |
| 20.3 | ±0.2 | 4.37 | ±0.04 | 12 |
| 20.8 | ±0.2 | 4.27 | ±0.04 | 79 |
| 22.0 | ±0.2 | 4.04 | ±0.04 | 35 |
| 22.9 | ±0.2 | 3.88 | ±0.03 | 10 |
| 23.5 | ±0.2 | 3.78 | ±0.03 | 39 |
| 23.8 | ±0.2 | 3.73 | ±0.03 | 30 |
| 24.0 | ±0.2 | 3.70 | ±0.03 | 18 |
| 24.5 | ±0.2 | 3.63 | ±0.03 | 37 |
| 25.0 | ±0.2 | 3.56 | ±0.03 | 19 |
| 25.8 | ±0.2 | 3.45 | ±0.03 | 29 |
| 26.0 | ±0.2 | 3.42 | ±0.03 | 31 |
| 26.7 | ±0.2 | 3.33 | ±0.02 | 61 |
| 27.7 | ±0.2 | 3.22 | ±0.02 | 27 |
| 29.2 | ±0.2 | 3.05 | ±0.02 | 29 |

TABLE 2

| | peak position | | coupling | number of |
|---|---|---|---|---|
| Protons | (ppm) | multiplicity | constant (Hz) | protons |
| CH₃ | 1.26 | triplet | 7 | obscured by |
| CH₃ | 1.30 | doublet | 8 | impurities |
| 3 × CH₂ | 1.42-1.57 | multiplet | — | 1 |
| | 1.72-1.95 | multiplet | — | 5 |
| CH₂CH₃ | 2.74 | quartet | 7 | 2 |
| 3 × CH₂N | 2.98-3.07 | multiplet | — | 2 |
| | 3.13-3.20 | multiplet | — | 1 |
| | 3.38-3.45 | multiplet | — | 1 |
| | 3.47-3.51 | multiplet | — | impurity |
| | 3.58-3.61 | multiplet | — | 1 |
| | 3.73-3.78 | multiplet | — | 1 |
| CH | 4.12-4.21 | multiplet | — | 1 |
| Aromatic | 7.40 | doublet | 8 | 2 |
| Aromatic | 8.02 | doublet | 8 | 2 |

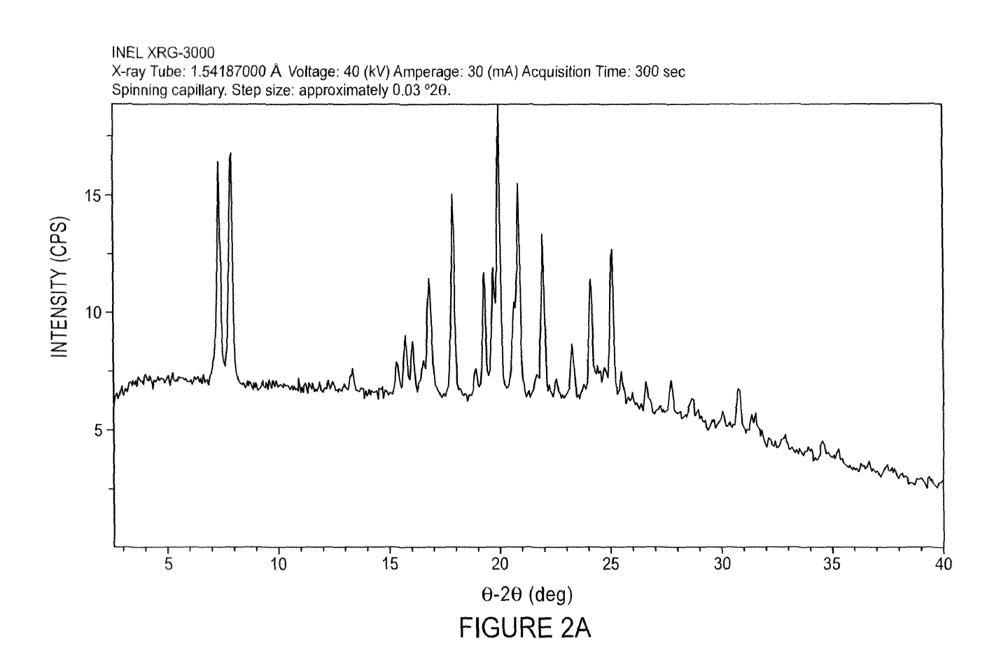

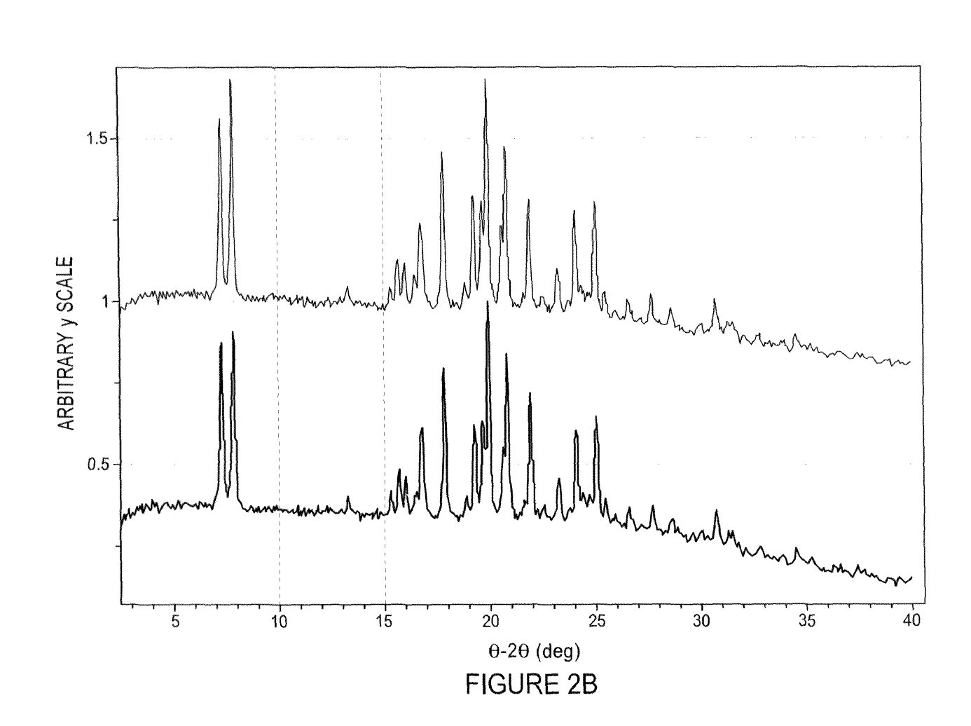

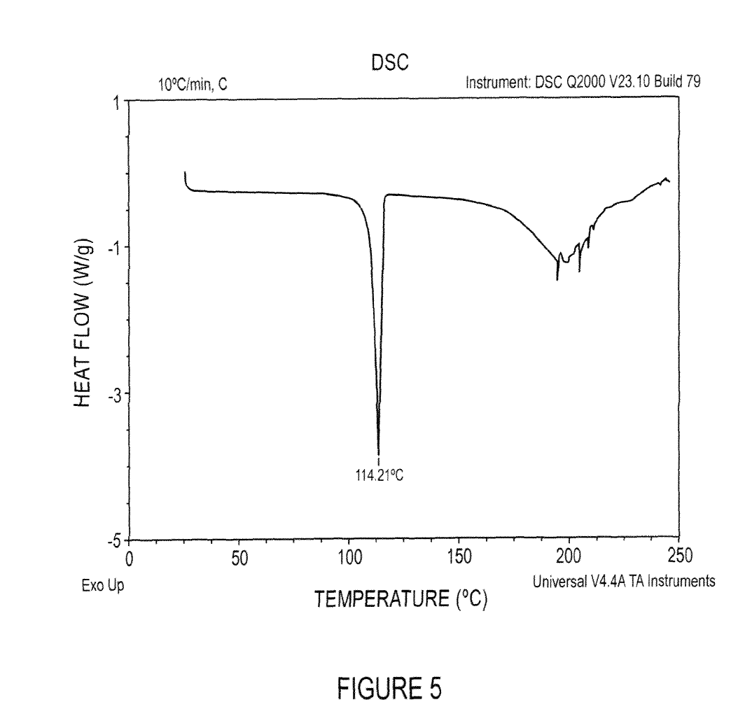

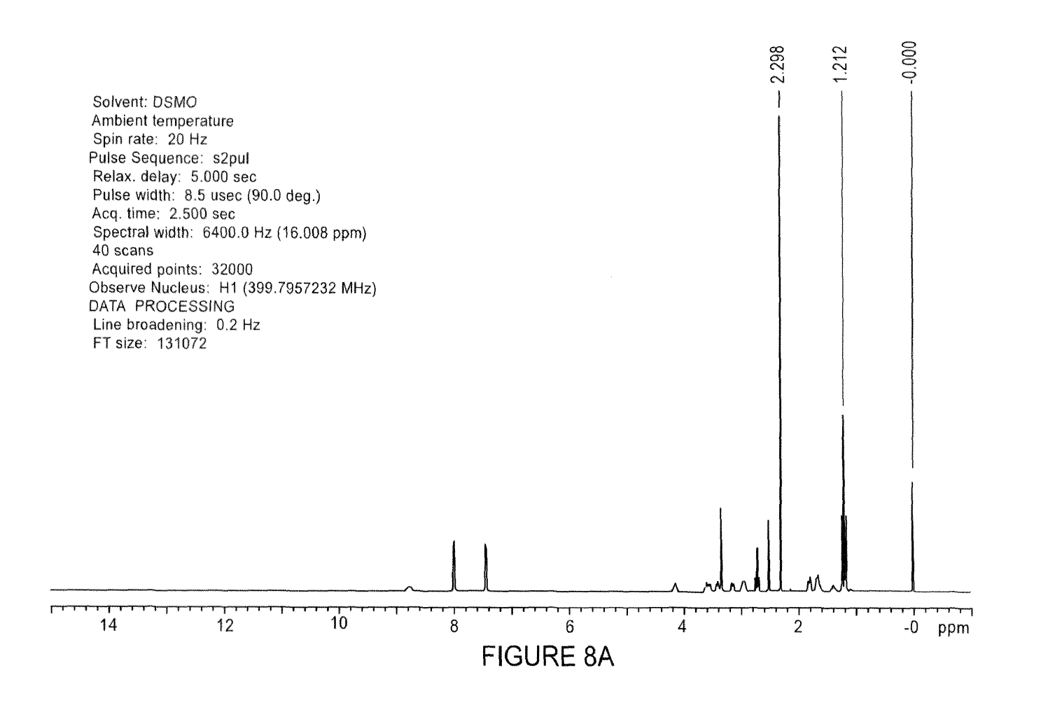

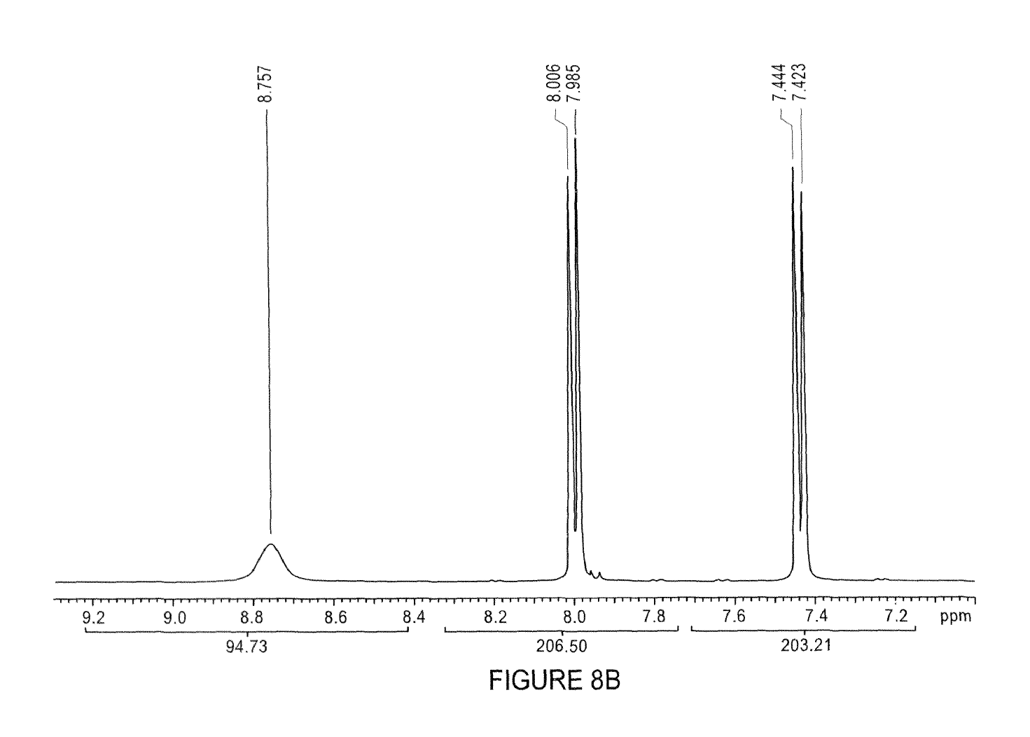

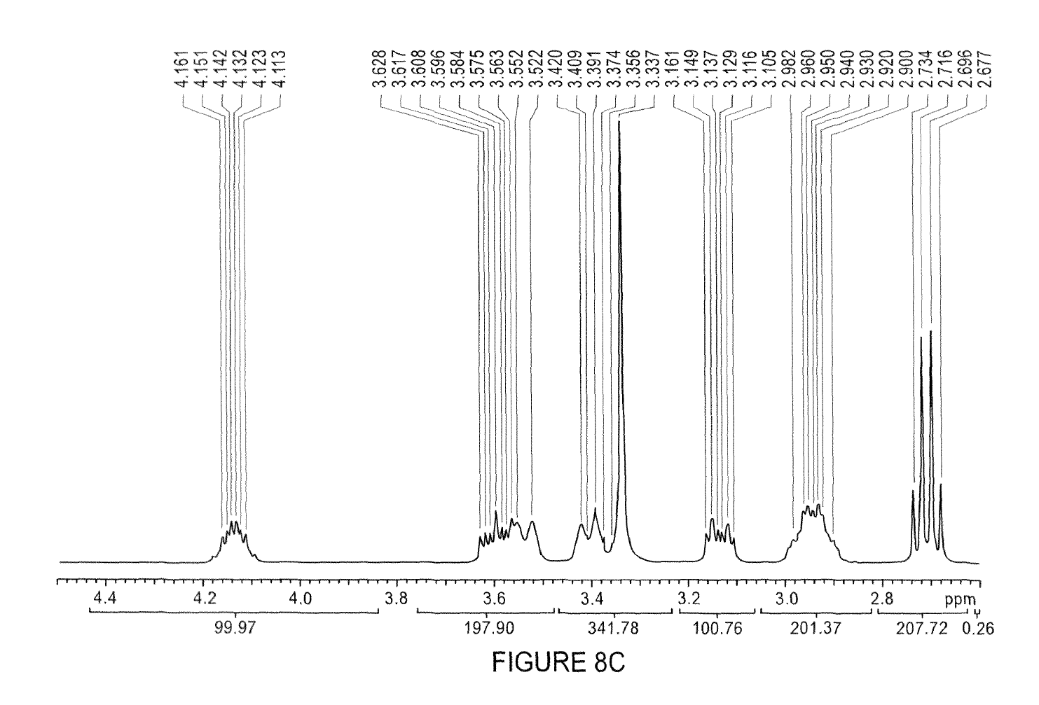

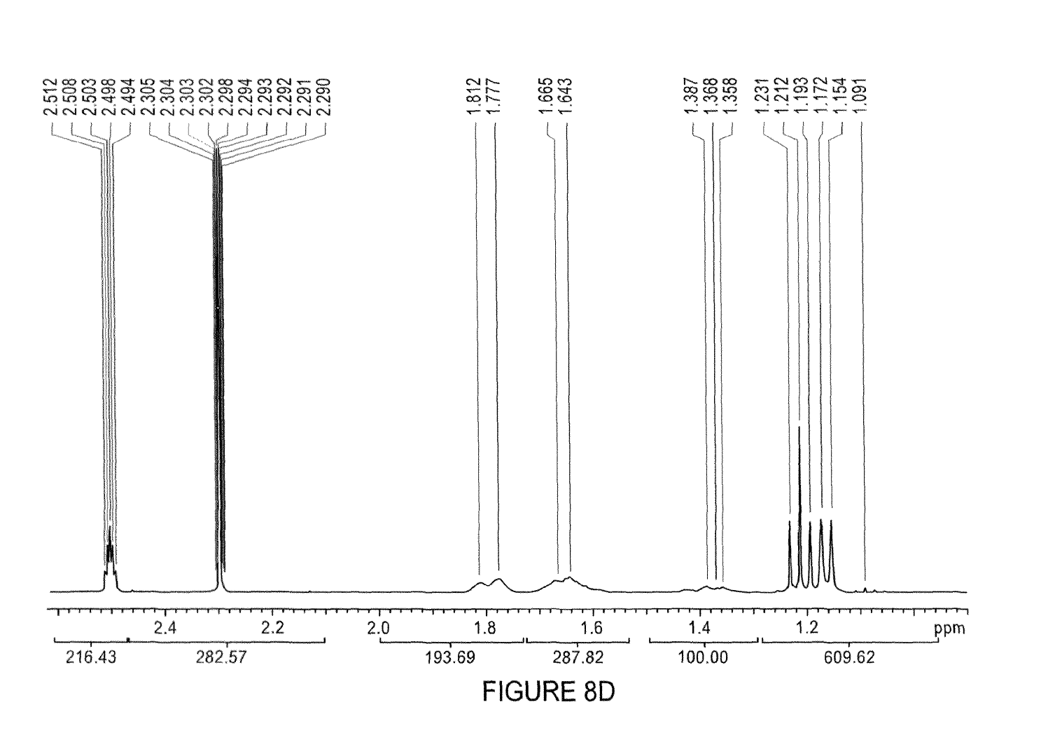

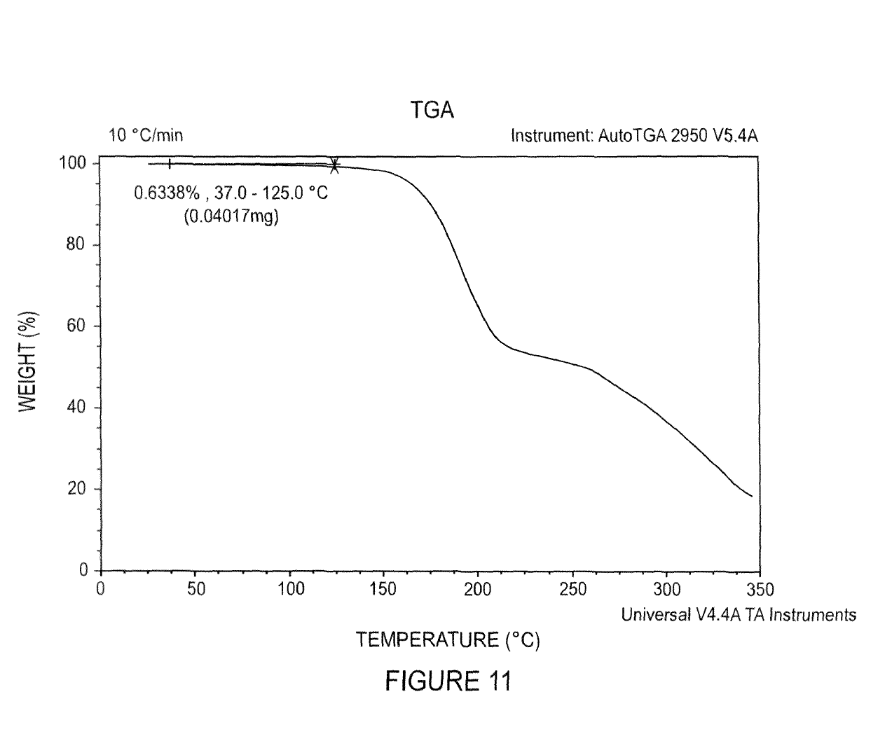

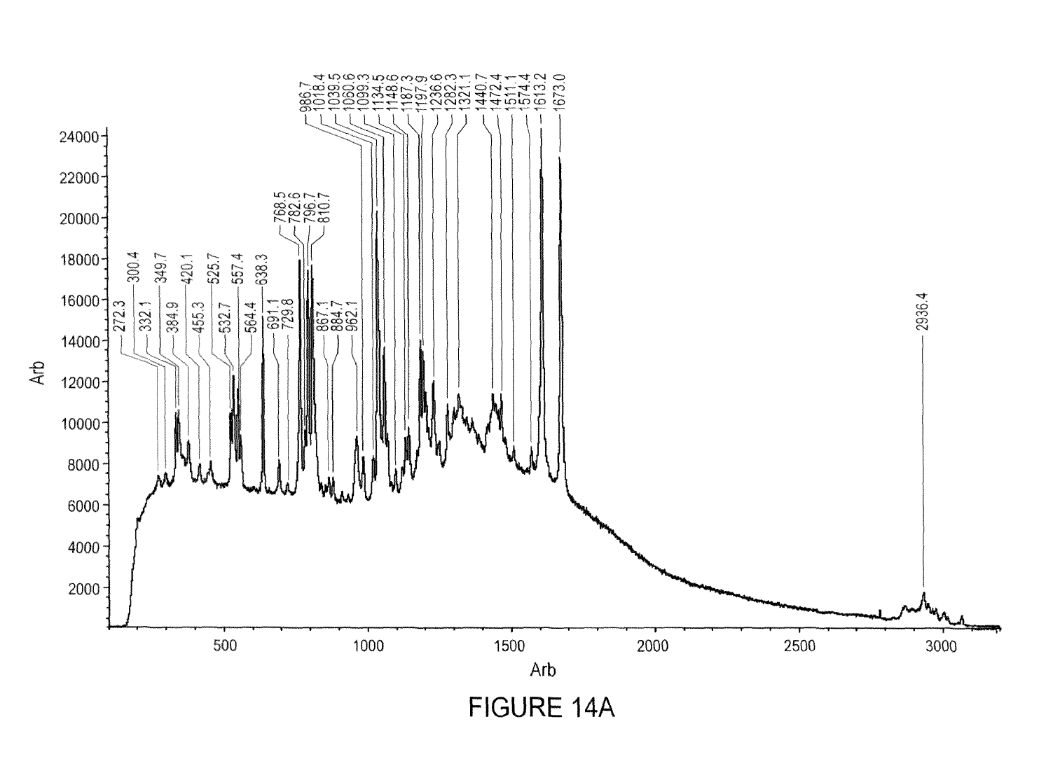

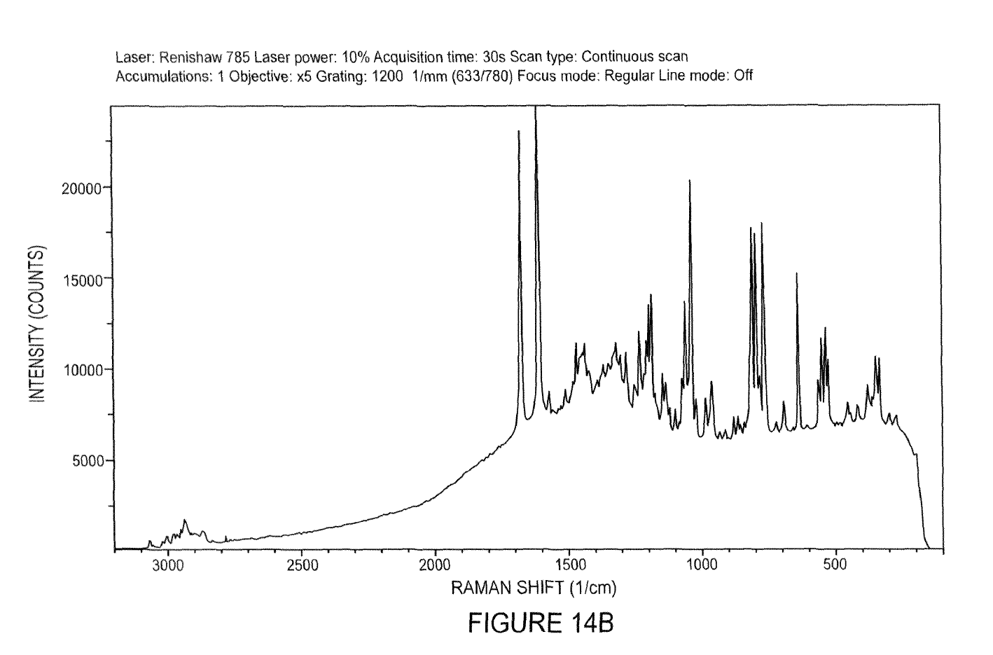

Substantially enantiopure crystalline eperisone mesylate is characterized by an XRPD pattern substantially as shown in FIG. 2A, a DSC thermogram substantially as shown in FIG. 5, an ¹H-NMR spectrum substantially as shown in FIGS. 8A-8D, a TGA profile substantially as shown in FIG. 11, and Raman spectra substantially as shown in FIGS. 14A and 14B. FIG. 2B demonstrates that (+)-eperisone mesylate and (−)-eperisone mesylate have substantially similar XRPD patterns. An exemplary listing of representative XRPD peaks of a substantially enantiopure crystalline eperisone mesylate salt according to an embodiment of the invention can be found in Table 3. An exemplary listing of representative NMR data, obtained in DMSO-d₆, can be found in Table 4.

TABLE 3

| Degrees 2θ | d spacing Å | Intensity % (I/Io) |
|---|---|---|
| 7.3 ± 0.2 | 12.10 ± 0.34 | 78 |
| 7.9 ± 0.2 | 11.18 ± 0.29 | 98 |
| 13.3 ± 0.2 | 6.65 ± 0.10 | 10 |
| 15.4 ± 0.2 | 5.75 ± 0.08 | 10 |
| 15.7 ± 0.2 | 5.64 ± 0.07 | 22 |
| 16.0 ± 0.2 | 5.53 ± 0.07 | 21 |
| 16.5 ± 0.2 | 5.37 ± 0.07 | 16 |
| 16.8 ± 0.2 | 5.27 ± 0.06 | 38 |
| 17.9 ± 0.2 | 4.95 ± 0.06 | 69 |
| 18.9 ± 0.2 | 4.69 ± 0.05 | 11 |
| 19.3 ± 0.2 | 4.59 ± 0.05 | 50 |
| 19.7 ± 0.2 | 4.50 ± 0.05 | 47 |
| 20.0 ± 0.2 | 4.43 ± 0.04 | 100 |
| 20.6 ± 0.2 | 4.31 ± 0.04 | 37 |
| 20.9 ± 0.2 | 4.25 ± 0.04 | 70 |
| 21.6 ± 0.2 | 4.11 ± 0.04 | 8 |
| 22.0 ± 0.2 | 4.04 ± 0.04 | 50 |
| 22.6 ± 0.2 | 3.93 ± 0.03 | 7 |
| 23.3 ± 0.2 | 3.81 ± 0.03 | 20 |
| 23.8 ± 0.2 | 3.73 ± 0.03 | 6 |
| 24.1 ± 0.2 | 3.69 ± 0.03 | 47 |
| 25.1 ± 0.2 | 3.54 ± 0.03 | 52 |
| 25.5 ± 0.2 | 3.49 ± 0.03 | 12 |
| 26.0 ± 0.2 | 3.42 ± 0.03 | 4 |
| 26.6 ± 0.2 | 3.35 ± 0.02 | 10 |
| 27.7 ± 0.2 | 3.22 ± 0.02 | 14 |
| 28.6 ± 0.2 | 3.12 ± 0.02 | 9 |

TABLE 4

| | peak position | | coupling | number of |
|---|---|---|---|---|
| Protons | (ppm) | multiplicity | constant (Hz) | protons |
| CHCH₃ | 1.16 | doublet | 7 | 6 |
| CH₂CH₃ | 1.21 | triplet | 8 | |
| 3 × CH₂ | 1.32-1.44 | multiplet | — | 1 |
| | 1.60-1.72 | multiplet | — | 3 |
| | 1.75-1.85 | multiplet | — | 2 |
| CH₃SO₃ | 2.30 | singlet | — | 3 |
| CH₂CH₃ | 2.71 | quartet | 8 | 2 |
| 3 × CH₂N | 2.90-2.98 | multiplet | — | 2 |
| | 3.11-3.16 | multiplet | — | 1 |
| | 3.34-3.42 | multiplet | — | obscured by water |
| | 3.52-3.63 | multiplet | — | 2 |
| CH | 4.11-4.16 | multiplet | — | 1 |
| aromatic | 7.43 | doublet | 8 | 2 |
| aromatic | 8.00 | doublet | 8 | 2 |
| NH | 8.76 | broad singlet | — | 1 |

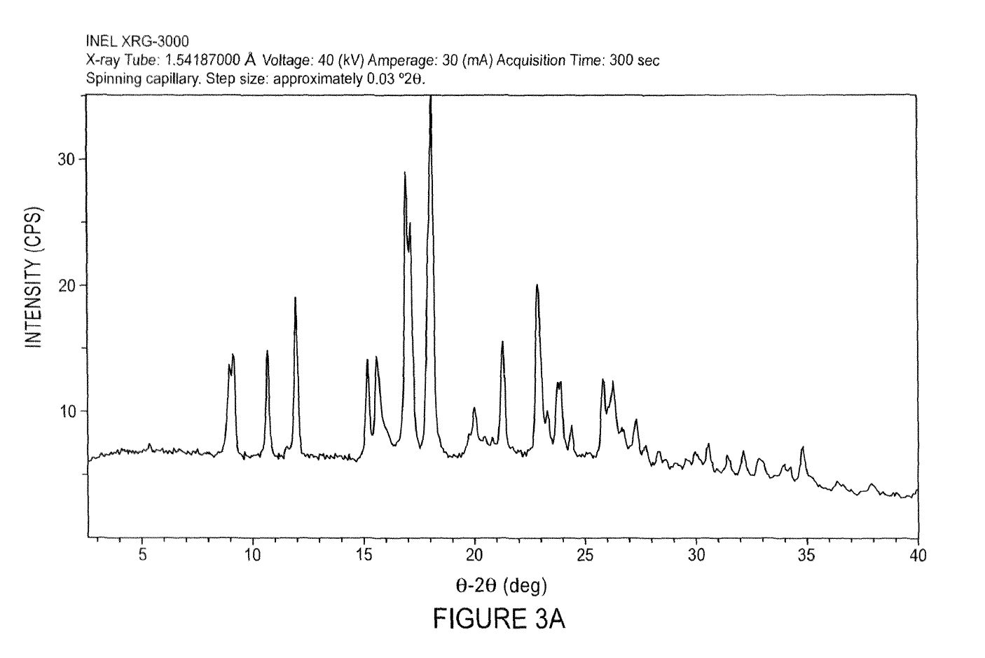

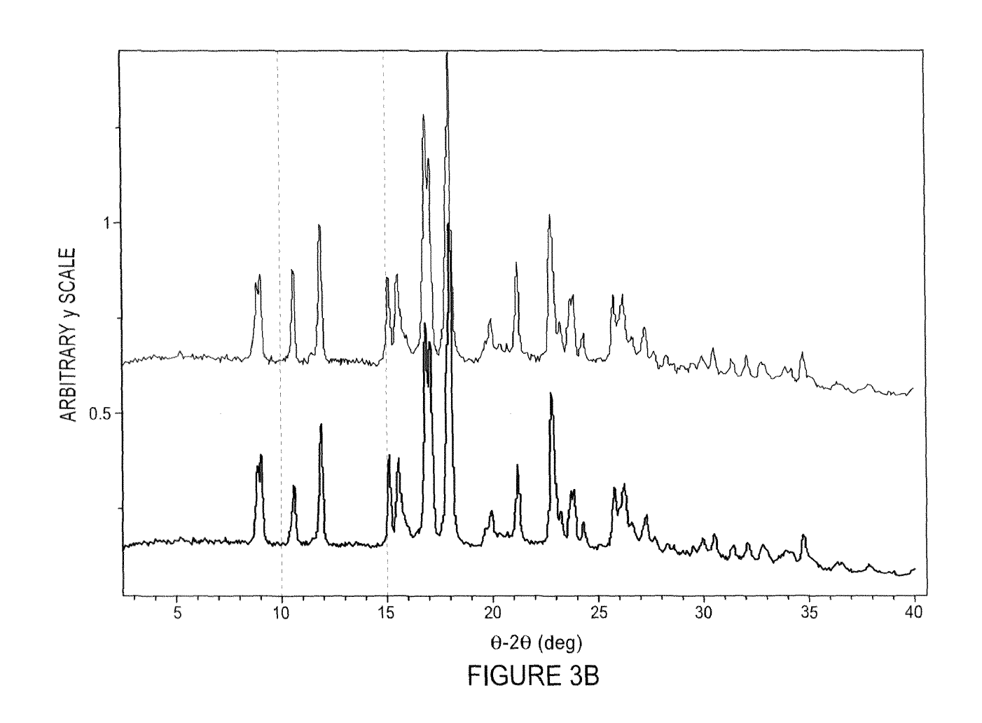

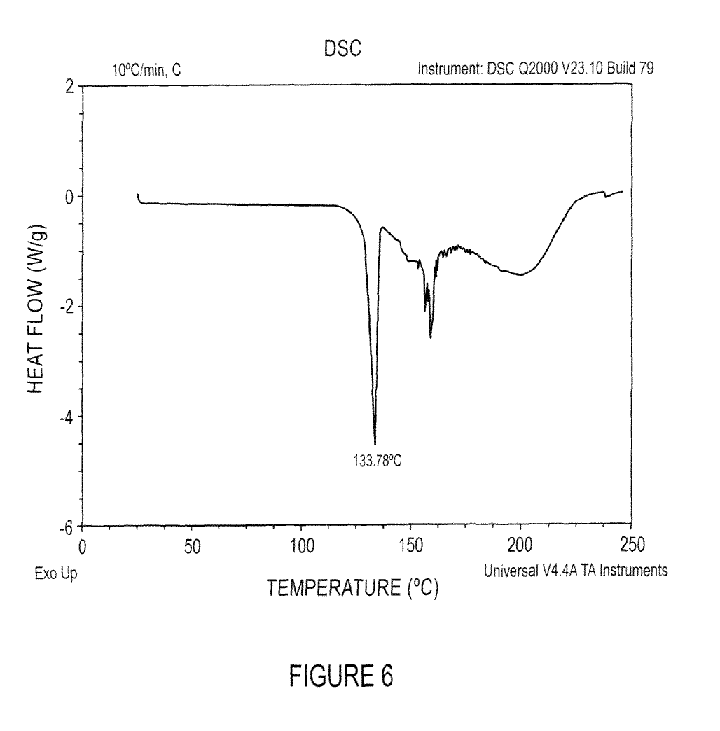

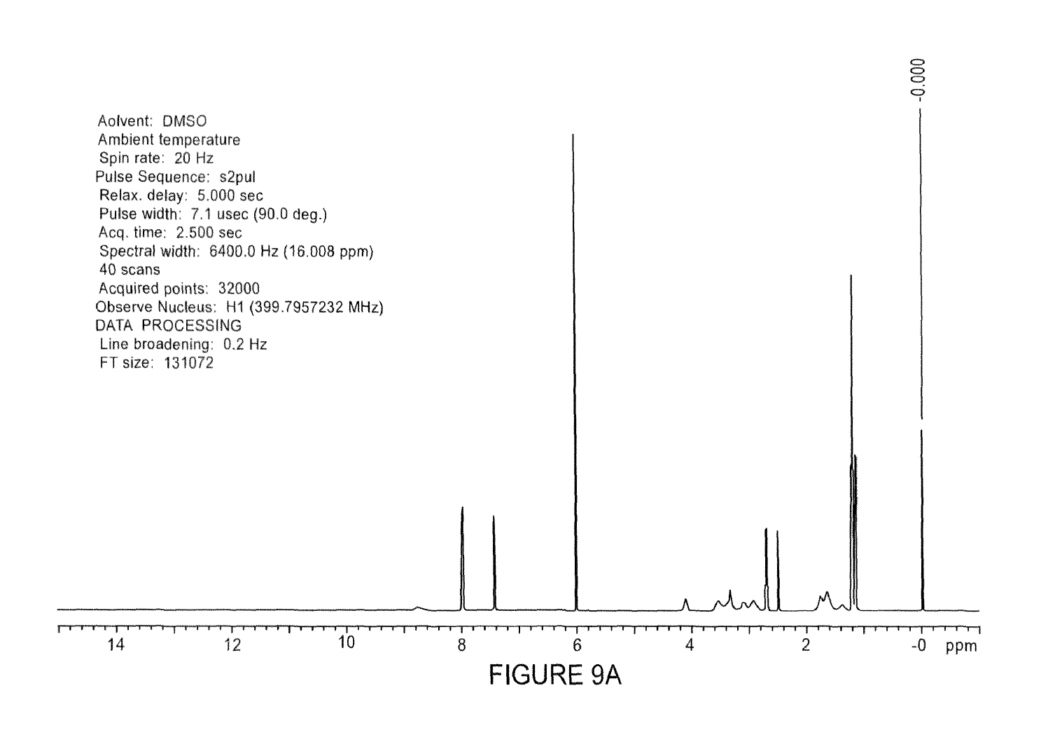

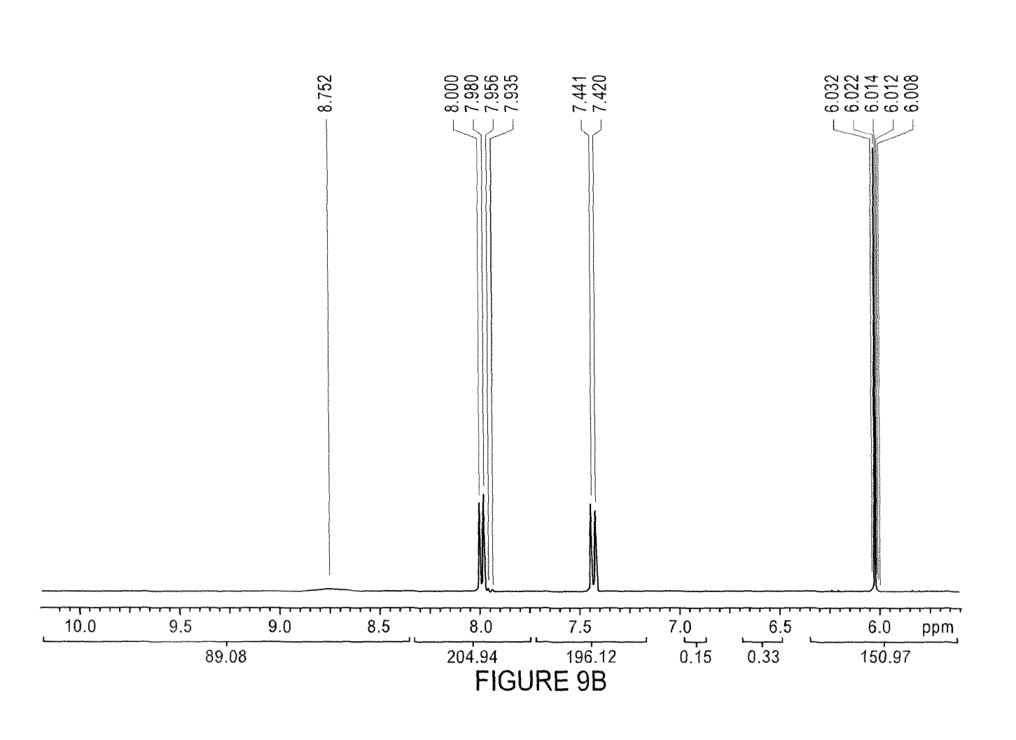

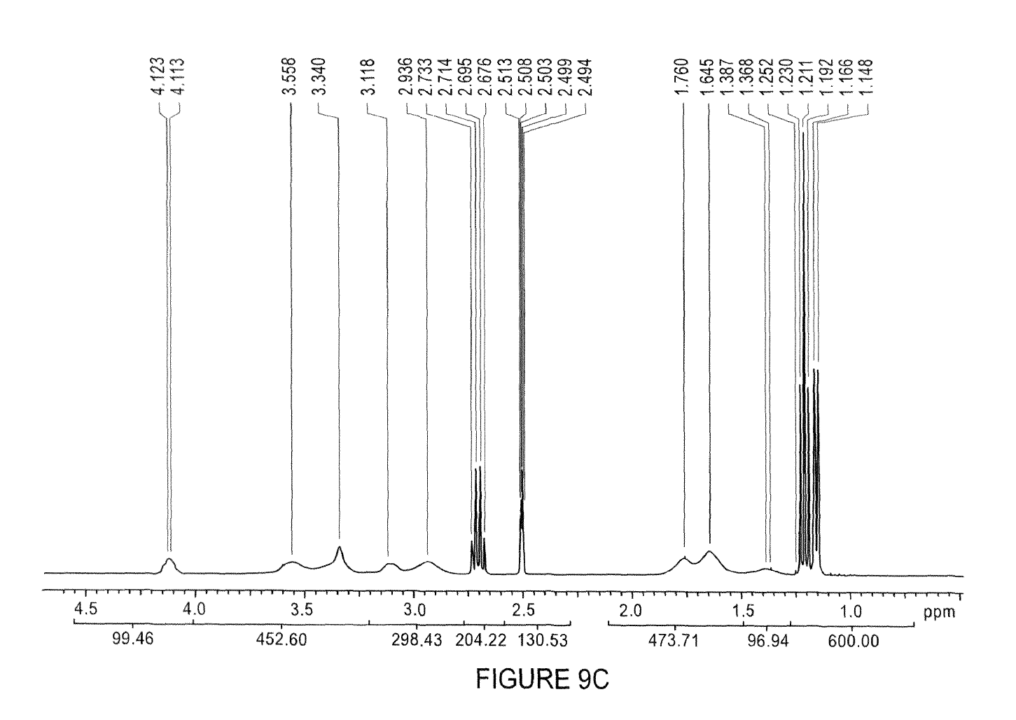

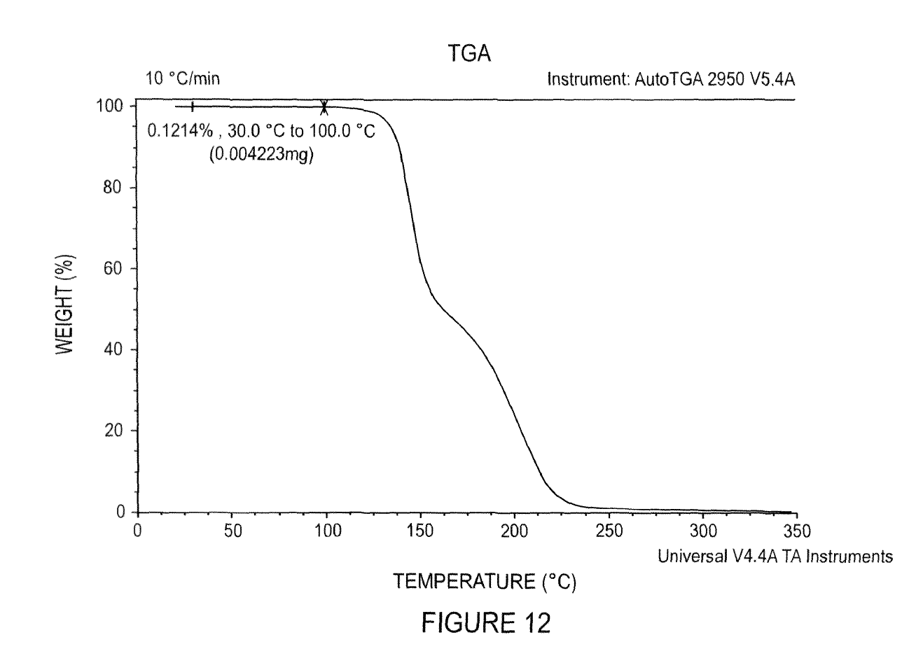

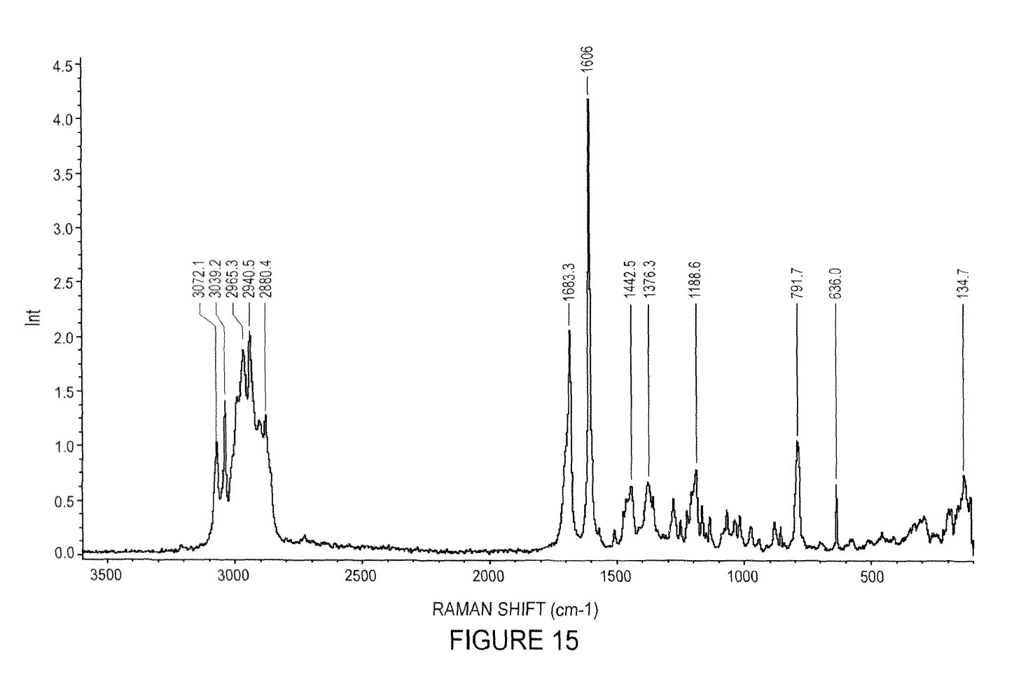

Substantially enantiopure crystalline eperisone maleate salt is characterized by an XRPD pattern substantially as shown in FIG. 3A, a DSC thermogram substantially as shown in FIG. 6, an ¹H-NMR spectrum substantially as shown in FIGS. 9A-9C, a TGA profile substantially as shown in FIG. 12, and a Raman spectrum substantially as shown in FIG. 15. FIG. 3B demonstrates that (+)-eperisone maleate and (−)-eperisone maleate have substantially similar XRPD patterns. An exemplary listing of representative XRPD peaks of a substantially enantiopure crystalline eperisone maleate salt according to an embodiment of the invention can be found in Table 5. An exemplary listing of representative NMR data, obtained in DMSO-d₆, can be found in Table 6.

TABLE 5

| Degrees 2θ | d spacing Å | Intensity (I/Io) |
|---|---|---|
| 8.9 ± 0.2 | 9.92 ± 0.23 | 26 |
| 9.0 ± 0.2 | 9.81 ± 0.22 | 29 |
| 10.6 ± 0.2 | 8.34 ± 0.16 | 30 |
| 11.9 ± 0.2 | 7.43 ± 0.13 | 45 |
| 15.1 ± 0.2 | 5.86 ± 0.08 | 28 |
| 15.5 ± 0.2 | 5.71 ± 0.07 | 29 |
| 16.9 ± 0.2 | 5.24 ± 0.06 | 79 |
| 17.0 ± 0.2 | 5.21 ± 0.06 | 65 |
| 18.0 ± 0.2 | 4.92 ± 0.05 | 100 |
| 19.9 ± 0.2 | 4.46 ± 0.04 | 14 |
| 21.2 ± 0.2 | 4.19 ± 0.04 | 32 |
| 22.8 ± 0.2 | 3.90 ± 0.03 | 48 |
| 23.2 ± 0.2 | 3.83 ± 0.03 | 13 |
| 23.8 ± 0.2 | 3.73 ± 0.03 | 21 |
| 24.3 ± 0.2 | 3.66 ± 0.03 | 8 |
| 25.8 ± 0.2 | 3.45 ± 0.03 | 23 |
| 26.2 ± 0.2 | 3.40 ± 0.03 | 23 |
| 27.2 ± 0.2 | 3.27 ± 0.02 | 13 |
| 27.7 ± 0.2 | 3.22 ± 0.02 | 6 |
| 28.3 ± 0.2 | 3.15 ± 0.02 | 5 |
| 29.9 ± 0.2 | 2.98 ± 0.02 | 6 |

TABLE 6

| | peak position | | coupling | number of |
|---|---|---|---|---|
| protons | (ppm) | multiplicity | constant (Hz) | protons |
| CHCH₃ | 1.16 | doublet | 7 | 6 |
| CH₂CH₃ | 1.21 | triplet | 8 | |
| 3 × CH₂ | 1.37 | broad multiplet | — | 1 |
| | 1.65 | broad multiplet | — | 5 |
| | 1.76 | broad multiplet | — | |
| CH₂CH₃ | 2.70 | quartet | 8 | 2 |
| 3 × CH₂N | 2.94 | broad multiplet | — | 3 |
| | 3.12 | broad multiplet | — | |
| | 3.34 | broad multiplet | — | obscured by |
| | 3.56 | broad multiplet | — | water |
| CH | 4.11 | broad multiplet | — | 1 |
| olefin | 6.02 | singlet | — | 2 |
| aromatic | 7.43 | doublet | 8 | 2 |
| aromatic | 7.99 | doublet | 8 | 2 |
| NH | 8.75 | broad singlet | — | 1 |

Pharmaceutical Compositions and Methods of Treatment and/or Prevention

The substantially enantiopure crystalline salt forms of eperisone according to various embodiments of the invention possess substantially the same pharmacological activity as racemic eperisone hydrochloride, and are useful for treating and/or preventing the discomfort, muscle spasm, stiffness, or myotonic conditions associated with painful musculoskeletal conditions, such as, for example, low back pain, neck pain, neck-shoulder-arm syndrome, scapulohumeral periarthritis, cervical spondylosis, and other musculoskeletal conditions; spasticity or spastic paralysis of neurological origin due to multiple sclerosis, spinal cord injury, traumatic brain injury, cerebral palsy, stroke or cerebrovascular disorder, spastic spinal paralysis, sequelae of surgical trauma (including, for example, cerebrospinal tumor), amyotrophic lateral sclerosis, spinocerebellar degeneration, spinal vascular disorders, subacute myelo-optico neuropathy (SMON) and other encephalomyelopathies, and other neurological conditions: primary dystonia; secondary dystonia; tension headache; fibromyalgia; chronic fatigue syndrome; muscle cramps; hypertension; and cancer.

The substantially enantiopure crystalline salt forms of eperisone according to various embodiments of the invention are also useful for treating and/or preventing disorders that arise from altered cell membrane excitability, including, for example, long QT syndrome, Brugada syndrome, heart arrhythmias, malignant hyperthermia, myasthenia, epilepsy, ataxia, migraine, Alzheimer's Disease, Parkinson's Disease, Huntington's Disease, schizophrenia, psychosis, bipolar disorder, hyperekplexia, neuropathic pain and pain associated with nervous system disorders such as, for example, painful diabetic neuropathy, postherpetic neuralgia, trigeminal neuralgia, complex regional pain syndrome I, complex regional pain syndrome II, ischemic neuropathy, phantom limb pain, chemotherapy-induced neuropathy, HIV-related neuropathy, AIDS-related neuropathy, neuropathic back pain, neuropathic neck pain, carpal tunnel syndrome, other forms of nerve entrapment or nerve compression pain, brachial plexus lesions, other peripheral nerve lesions, neuropathic cancer pain, vulvodynia, central neuropathic pain, pain due to multiple sclerosis, post-stroke pain, Parkinson's Disease related central pain, postoperative chronic pain, Guillain-Barre syndrome (GBS), Charcot-Marie-Tooth (CMT) disease, idiopathic peripheral neuropathy, alcoholic neuropathy, other types of neuropathic pain, and other nervous system disorders that have pain as an attendant sign and/or symptom.

The substantially enantiopure crystalline salt forms of eperisone according to various embodiments of the invention are also useful for treating and/or preventing non-neuropathic pain of various etiologies, including, by way of example only, inflammatory pain, cancer pain, pain resulting from traumatic injury, post-operative pain, dysmenorrhea, osteoarthritis, rheumatoid arthritis, psoriatic arthritis, gout, tendonitis pain, bursitis pain, sports injury-related pain, sprains, strains, pain of osteoporosis, ankylosing spondylitis, headache, temporomandibular joint pain, interstitial cystitis, myofascial pain syndrome, pain of irritable bowel syndrome, idiopathic chronic pain, and visceral pain.

By use of the term “treating” or “alleviating” it is meant decreasing the symptoms, markers, and/or any negative effects of a condition in any appreciable degree in a patient who currently has the condition, and by “preventing” it is meant preventing entirely or preventing to some extent, such as, for example, by delaying the onset or lessening the degree to which a patient develops the condition.

As discussed, additional embodiments of the invention relate to pharmaceutical compositions comprising a therapeutically effective amount of one or more substantially enantiopure crystalline salt forms of eperisone according to various embodiments of the invention, and a pharmaceutically acceptable carrier or excipient. The substantially enantiopure crystalline salt forms of eperisone according to various embodiments of the invention have the same or similar pharmaceutical activity as previously reported for racemic eperisone hydrochloride. Pharmaceutical compositions for the treatment and/or prevention of the enumerated conditions or disorders may contain some amount, for example a therapeutically effective amount, of one or more of the substantially enantiopure crystalline salt forms of eperisone described herein, as appropriate, e.g. for treatment of a patient with the particular condition or disorder. As a further example, the amount of the one or more substantially enantiopure crystalline salt forms of eperisone in the pharmaceutical compositions may likewise be lower than a therapeutically effective amount, and may, for example, be in the composition in conjunction with another compound or form of eperisone which, when combined, are present in a therapeutically effective amount. A “therapeutically effective amount” as described herein refers to an amount of a therapeutic agent sufficient to treat, alleviate, and/or prevent a condition treatable and/or preventable by administration of a composition of the invention, in any degree. That amount can be an amount sufficient to exhibit a detectable therapeutic or preventative or ameliorative effect, and can be determined by routine experimentation by those of skill in the art. The effect may include, for example, treatment, alleviation, and/or prevention of the conditions listed herein. The actual amount required, e.g. for treatment of any particular patient, will depend upon a variety of factors including the disorder being treated and/or prevented; its severity; the specific pharmaceutical composition employed; the age, body weight, general health, gender, and diet of the patient; the mode of administration; the time of administration; the route of administration; the rate of excretion of eperisone; the duration of the treatment; any drugs used in combination or coincidental with the specific compound employed; and other such factors well known in the medical arts. These factors are discussed in Goodman and Gilman's “The Pharmacological Basis of Therapeutics”, Tenth Edition, A. Gilman, J. Hardman and L. Limbird, eds., McGraw-Hill Press, 155-173, 2001.

A pharmaceutical composition according to various embodiments of the invention may be any pharmaceutical form which contains one or more substantially enantiopure crystalline salt forms of eperisone according to various embodiments of the invention. Depending on the type of pharmaceutical composition, the pharmaceutically acceptable carrier may be chosen from any one or a combination of carriers known in the art. The choice of the pharmaceutically acceptable carrier depends upon the pharmaceutical form and the desired method of administration to be used. For a pharmaceutical composition according to various embodiments of the invention, that is one having one or more of the substantially enantiopure crystalline salt forms of eperisone described herein, a carrier may be chosen that maintains the crystalline salt form and/or the substantially enantiopure form. In other words, the carrier, in some embodiments, will not substantially alter the crystalline form or the enantiomeric purity of the forms of eperisone described herein. In certain embodiments, the carrier will similarly not be otherwise incompatible with eperisone itself, crystalline salts of eperisone, or substantially enantiopure crystalline salt forms of eperisone according to various embodiments of the invention, such as by producing any undesirable biological effect or otherwise interacting in a deleterious manner with any other component(s) of the pharmaceutical composition.

The pharmaceutical compositions according to various embodiments of the invention are optionally formulated in unit dosage form for ease of administration and uniformity of dosage. A “unit dosage form” refers to a physically discrete unit of therapeutic agent appropriate for the patient to be treated. It will be understood, however, that the total daily dosage of the substantially enantiopure crystalline salt forms of eperisone according to various embodiments of the invention and pharmaceutical compositions thereof will be decided by the attending physician within the scope of sound medical judgment using known methods.

Because the substantially enantiopure crystalline salt forms of eperisone may be more easily maintained during preparation, solid dosage forms are a preferred form for the pharmaceutical composition of the invention. Solid dosage forms for oral administration may include, for example, capsules, tablets, pills, powders, and granules. In one exemplary embodiment, the solid dosage form is a tablet. The active ingredient may be contained in a solid dosage form formulation that provides quick release, sustained release, or delayed release after administration to the patient. In such solid dosage forms, the active compound may be mixed with at least one inert, pharmaceutically acceptable carrier, such as, for example, sodium citrate or dicalcium phosphate. The solid dosage form may also include one or more of various additional ingredients, including, for example: a) fillers or extenders such as, for example, starches, lactose, sucrose, glucose, mannitol, and silicic acid; b) binders such as, for example, carboxymethylcellulose, alginates, gelatin, polyvinylpyrrolidinone, sucrose, and acacia; c) humectants such as, for example, glycerol; d) disintegrating agents such as, for example, agar, calcium carbonate, potato or tapioca starch, alginic acid, certain silicates, and sodium carbonate; e) dissolution retarding agents such as, for example, paraffin; f) absorption accelerators such as, for example, quaternary ammonium compounds; g) wetting agents such as, for example, cetyl alcohol and glycerol monostearate; h) absorbents such as, for example, kaolin and bentonite clay; and i) lubricants such as, for example, talc, calcium stearate, magnesium stearate, solid polyethylene glycols, and sodium lauryl sulfate. The solid dosage forms may also comprise buffering agents. They may optionally contain opacifying agents and can also be of a composition that they release the active ingredient(s) only, or preferentially, in a certain part of the intestinal tract, optionally, in a delayed manner. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Solid dosage forms of pharmaceutical compositions according to various embodiments of the invention can also be prepared with coatings and shells such as enteric coatings and other coatings well known in the pharmaceutical formulating art.

The substantially enantiopure crystalline salt forms of eperisone according to various embodiments of the invention can be, in one exemplary embodiment, administered in a solid micro-encapsulated form with one or more carriers as discussed above. Microencapsulated forms may also be used in soft and hard-filled gelatin capsules with carriers such as lactose or milk sugar as well as high molecular weight polyethylene glycols and the like.

The substantially enantiopure crystalline salt forms of eperisone according to various embodiments of the invention may also be used in the preparation of non-solid formulations, e.g., injectables and patches, of eperisone. Such non-solid formulations are known in the art. In certain formulations, such as a non-solid formulation, the enantiomeric purity or the crystalline salt form may, in certain exemplary embodiments, not be maintained. For example, the crystalline salt form may be dissolved in a liquid carrier. In this case, the substantially enantiopure crystalline salt forms of eperisone according to various embodiments of the invention may represent intermediate forms of eperisone used in the preparation of the non-solid formulation. The substantially enantiopure crystalline salt forms of eperisone according to various embodiments of the invention may provide advantages of handling stability and purity to the process of making such formulations.

In addition, the substantially enantiopure crystalline salt forms of eperisone according to various embodiments of the invention are also useful for administration in combination with other analgesic medication classes, such as strong and weak opioids, NSAIDs, COX-2 inhibitors, acetaminophen, other anti-inflammatories, tricyclic antidepressants, anticonvulsant agents, voltage gated calcium channel blockers, N-type calcium channel blockers, other calcium channel modulators, SNRIs and other monoamine reuptake inhibitors, sodium channel blockers, NK-1 antagonists, NMDA antagonists, AMPA antagonists, other glutamate modulators, GABA modulators, CRMP-2 modulators, TRPV1 agonists, cannabinoids, potassium channel openers, alpha adrenergic agonists, adenosine agonists, nicotinic agonists, p38 MAP kinase inhibitors, corticosteroids, and other analgesic drug classes, and may have a useful dose-sparing effect of lowering the required dosage of the medication used in combination with a substantially enantiopure crystalline salt form of eperisone according to various embodiments of the invention. The substantially enantiopure crystalline salt forms of eperisone according to various embodiments of the invention are therefore also useful for treating or preventing complications or side effects arising from usage of other analgesic medications, including problems with opioids such as dependency, constipation, and respiratory depression. Opioid pain medications can either inhibit or excite the CNS, although it is considered that inhibition is more common. Patients with depressed CNS functions may feel varying levels of drowsiness, lightheadedness, euphoria or dysphoria, or confusion. NSAID pain medications can also induce negative side effects, such as gastrointestinal toxicity or bleeding, renal toxicity, and cardiovascular toxicity. Side effects of other analgesic classes can include sedation, dizziness, anticholinergic effects, dependency, hypotension, and various other adverse effects. These analgesic-induced side effects can manifest themselves when the dosage is increased. Decreasing the dosage of an analgesic or changing medications often helps to decrease the rate or severity of these analgesic-induced side effects. It is possible that a therapeutic amount of a substantially enantiopure crystalline salt form of eperisone according to various embodiments of the invention in combination with a pain agent will reduce the risk of such side effects by reducing the required dosage of the other agent used in combination.

The invention also relates to the treatment and/or prevention of various disorders and/or conditions such as those discussed above, including, for example, pathological muscle contracture, myotonic conditions, spastic paralysis or spasticity caused by various neurologic conditions, and various types of pain and pathological muscle tension. The invention provides a method for treating and/or preventing such disorders and/or conditions by administering to mammals, such as a human, one or more of the substantially enantiopure crystalline salt forms of eperisone as described herein, or a pharmaceutical composition containing the same, in an amount sufficient to treat and/or prevent a condition treatable and/or preventable by administration of a composition of the invention. That amount is the amount sufficient to exhibit any detectable therapeutic and/or preventative or ameliorative effect. The effect may include, for example, treatment and/or prevention of the conditions listed herein. These substantially enantiopure crystalline salt forms of eperisone and pharmaceutical compositions containing them may, according to various embodiments of the invention, be administered using any amount, any form of pharmaceutical composition, and any route of administration effective, e.g. for treatment and/or prevention, all of which are easily determined by those of skill in the art through routine experimentation. After formulation with an appropriate pharmaceutically acceptable carrier in a desired dosage, as known by those of skill in the art, the pharmaceutical compositions can be administered to humans and other mammals by any known method, such as, for example, orally, rectally, or topically (such as by powders or other solid form-based topical formulations). In certain embodiments, the substantially enantiopure crystalline salt forms of eperisone according to various embodiments of the invention may be administered at dosage levels ranging from about 0.001 mg/kg to about 50 mg/kg, from about 0.01 mg/kg to about 25 mg/kg, or from about 0.1 mg/kg to about 10 mg/kg of subject body weight per day, one or more times a day, to obtain the desired therapeutic effect. It will also be appreciated that dosages smaller than about 0.001 mg/kg or greater than about 50 mg/kg (for example, ranging from about 50 mg/kg to about 100 mg/kg) can also be administered to a subject in certain embodiments of the invention. As discussed above, the amount required for a particular patient will depend upon a variety of factors including the disorder being treated and/or prevented; its severity; the specific pharmaceutical composition employed; the age, body weight, general health, gender, and diet of the patient: the mode of administration; the time of administration; the route of administration; and the rate of excretion of eperisone; the duration of the treatment; any drugs used in combination or coincidental with the specific compound employed; and other such factors well known in the medical arts. And, as also discussed, the pharmaceutical composition of the substantially enantiopure crystalline salt forms of eperisone as described herein may be administered as a unit dosage form.

Although the present invention herein has been described with reference to various exemplary embodiments, it is to be understood that these embodiments are merely illustrative of the principles and applications of the present invention. Those having skill in the art would recognize that a variety of modifications to the exemplary embodiments may be made, without departing from the scope of the invention.

Moreover, it should be understood that various features and/or characteristics of differing embodiments herein may be combined with one another. It is therefore to be understood that numerous modifications may be made to the illustrative embodiments and that other arrangements may be devised without departing from the scope of the invention.

Furthermore, other embodiments of the invention will be apparent to those skilled in the art from consideration of the specification and practice of the invention disclosed herein. It is intended that the specification and examples be considered as exemplary only, with a scope and spirit being indicated by the claims.

### EXAMPLES

### Example 1

### Preparation of Substantially Enantiopure Crystalline Hydrochloride Salts of Eperisone

Racemic eperisone hydrochloride was separated into substantially enantiopure fractions of eperisone free base using chiral chromatography. The isocratic supercritical fluid chromatography method used a mobile phase composed of liquid CO₂ with a 5% cosolvent mixture of 50:50 methanol:isopropanol containing 2% isopropylamine. The column was a Chiralpak AD-H in a 3.0×25 cm format with a total mobile phase flow of 80 g/minute. Chromatography of 51.1 g of racemic eperisone hydrochloride afforded solutions of two fractions (Fraction 1 was earlier eluting; Fraction 2 was later eluting). Each solution was dried by rotary evaporation to give solids of each substantially pure enantiomer as the free base. Contaminating isopropylamine was removed from each fraction by adding acetonitrile to the solid and drying by rotary evaporation to remove the acetonitrile-isopropylamine azeotrope and acetonitrile. Each fraction consisted of white, waxy solids, which were dissolved in acetonitrile. Each acetonitrile solution was cooled with stirring in an ice bath and treated with a slowly bubbled stream of hydrochloride gas for 5 minutes. Rotary evaporation to remove acetonitrile yielded substantially pure, crystalline eperisone hydrochloride of each enantiomer as white powders. The yield of each fraction and enantiomeric purity of each fraction as determined by chiral HPLC are shown in the following table:

| Fraction | Mass Recovered | Enantiomeric Purity |
|---|---|---|
| Fraction 1 (1.22 min) | 10.1 g | 97.1% |
| Fraction 2 (1.42 min) | 13.4 g | 95.8% |

Analytical data were obtained on the final products: the XRPD patterns were as shown in FIGS. 1A and 1B, the DSC thermogram was as shown in FIG. 4, the ¹H-NMR spectrum was as shown in FIGS. 7A-7G, the TGA profile was as shown in FIG. 10, and the Raman spectra were as shown in FIGS. 13A and 13B. In acetonitrile, the solubility of the substantially enantiopure crystalline eperisone hydrochloride was determined to be greater than 11 mg/mL, calculated based on the total solvent used to give a solution. The optical rotation of substantially enantiopure eperisone hydrochloride fraction F1 was measured and the specific rotation calculated: [α]_{D}²⁵+14.95 deg cm² g⁻¹ (c 1.05, acetonitrile). The optical rotation of substantially enantiopure eperisone hydrochloride fraction F2 was measured and the specific rotation calculated: [α]_{D}²⁵−15.89 deg cm² g⁻¹ (c 1.05, acetonitrile).

### Example 2

### Preparation of a Substantially Enantiopure Crystalline Mesylate Salt of Eperisone

A mixture of 1.0 mL of methanesulfonic acid and 3.0 mL of diethyl ether was shaken to give a clear, colorless solution. Substantially enantiopure epalrestat free base, which was prepared from substantially enantiopure epalrestat hydrochloride Fraction 2 of Example 1, was dissolved in diethyl ether to give a solution containing 272 mg/mL of free base. In a nitrogen box, a 20-mL scintillation vial was placed in a crystallization dish containing dry ice. The vial was charged with 130 μL of the methanesulfonic acid solution (0.501 mmol acid) and 478 μL of the substantially enantiopure epalrestat free base solution (0.501 mmol of base). The vial was placed on a rotating wheel inside a −20° C. freezer for approximately 1 hour. The milky white slurry was filtered on a Magna 0.22-μm nylon membrane inside a Millipore Swinnex filter body. Diethyl ether (1 mL) was added to the mother liquor, and the resulting mixture was used to rinse the parent vial before filtering on top of the previously collected solids. The parent vial was rinsed with three 1-mL portions of diethyl ether, each time filtering the ether on top of the previously collected solids. The white solids were transferred back to the original vial and dried under a flow of nitrogen gas for 15 minutes to give 86.6 mg (49% yield) of substantially enantiopure eperisone mesylate. Optical microscopy indicated the solids to be opaque and birefringent. Analytical data were obtained on the final product: the XRPD pattern was as shown in FIG. 2A, the DSC thermogram was as shown in FIG. 5, the ¹H-NMR spectrum was as shown in FIGS. 8A-8D, the TGA profile was as shown in FIG. 11, and the Raman spectra were as shown in FIGS. 14A and 14B. HPLC analysis showed that the final product had a higher enantiomeric purity (98%) than that of the starting substantially enantiopure epalrestat hydrochloride.

### Example 3

### Preparation of a Substantially Enantiopure Crystalline Mesylate Salt of Eperisone

A mixture of 100 μL of methanesulfonic acid and 300 μL of diethyl ether was prepared, giving a clear solution. A solution was made by dissolving 342 mg of substantially enantiopure epalrestat free base, which was prepared from substantially enantiopure epalrestat hydrochloride Fraction 1 of Example 1, in 1.0 mL of diethyl ether. In a nitrogen bag, a 20-mL amber glass vial was placed on dry ice and charged with 17.5 μL of the methanesulfonic acid solution (0.067 mmol acid) and 50 μL of the substantially enantiopure epalrestat free base solution (0.066 mmol of base). The resulting mixture clouded immediately and oil was deposited on the bottom of the vial. The oil began to crystallize into needles. The vial was transferred to a freezer for approximately 30 minutes, at which time most of the oil had crystallized. The liquid was drawn off with a pipette and the remaining solids were washed with three 1-mL portions of ether. The solids were left in the original vial and dried under a stream of nitrogen gas for a few hours. XRPD data were obtained on the final product, and were as shown in FIG. 2B. HPLC analysis showed that the final product had a higher enantiomeric purity (99%) than that of the starting substantially enantiopure epalrestat hydrochloride. The optical rotation of the obtained substantially enantiopure eperisone mesylate was measured and the specific rotation calculated: [α]_{D}²⁷+19.29 deg cm² g⁻¹ (c 0.71, acetonitrile).

### Example 4

### Preparation of a Substantially Enantiopure Crystalline Maleate Salt of Eperisone

A solution was made by mixing 205 mg (0.790 mmol) of substantially enantiopure eperisone free base, which was prepared from substantially enantiopure epalrestat hydrochloride Fraction 1 of Example 1, and 2.0 mL of diethyl ether in a 20-mL scintillation vial. Maleic acid was dissolved in tetrahydrofuran to give a solution containing 50.6 mg/mL of acid. The base solution was treated drop wise with 1.82 mL (0.793 mmol of maleic acid) of the acid solution. A white precipitate formed. The slurry was agitated for approximately 15 hours at about −15° C. and filtered at ambient temperature on a Magna 0.22-μm nylon membrane inside a Millipore Swinnex filter body. The solids were dried in a vacuum oven (23-24° C., about 30 in. Hg vacuum) for approximately 10 hours to give 231 mg (78% yield) of substantially enantiopure eperisone maleate. Optical microscopy indicated splinter aggregates that exhibited birefringence and extinction. Analytical data were obtained on the final product: the XRPD pattern was as shown in FIG. 3A, the DSC thermogram was as shown in FIG. 6, the ¹H-NMR spectrum was as shown in FIGS. 9A-9C, the TGA profile was as shown in FIG. 12, and the Raman spectrum was as shown in FIG. 15. HPLC analysis showed that the final product had a high enantiomeric excess.

### Example 5

### Preparation of a Substantially Enantiopure Crystalline Maleate Salt of Eperisone

A solution was made by mixing 205 mg (0.790 mmol) of substantially enantiopure eperisone free base, which was prepared from substantially enantiopure epalrestat hydrochloride Fraction 2 of Example 1, and 2.0 mL of diethyl ether in a 20-mL scintillation vial. Maleic acid was dissolved in tetrahydrofuran to give a solution containing 50.6 mg/mL of acid. The base solution was treated drop wise with 1.81 mL (0.789 mmol of maleic acid) of the acid solution. A white precipitate formed. The slurry was agitated for approximately 15 hours at about −15° C. and filtered at ambient temperature on a Magna 0.22-μm nylon membrane inside a Millipore Swinnex filter body. The solids were dried in a vacuum oven (23-24° C., about 30 in. Hg vacuum) for approximately 10 hours to give 217 mg (73% yield) of substantially enantiopure eperisone maleate. Analytical data were obtained on the final product: the XRPD pattern was as shown in FIG. 3B, and the DSC thermogram was substantially as shown in FIG. 6. HPLC analysis showed that the final product had a high enantiomeric excess.

1. A substantially enantiopure crystalline hydrochloride salt of (2RS)-1-(4-Ethylphenyl)-2-methyl-3-piperidin-1-ylpropan-1-one.

2. A substantially enantiopure crystalline mesylate salt of (2RS)-1-(4-Ethylphenyl)-2-methyl-3-piperidin-1-ylpropan-1-one.

3. A substantially enantiopure crystalline maleate salt of (2RS)-1-(4-Ethylphenyl)-2-methyl-3-piperidin-1-ylpropan-1-one.

4-6. (canceled)

7. A pharmaceutical composition comprising the substantially enantiopure crystalline hydrochloride salt of (2RS)-1-(4-Ethylphenyl)-2-methyl-3-piperidin-1-ylpropan-1-one according to claim 1.

8. A pharmaceutical composition comprising the substantially enantiopure crystalline mesylate salt of (2RS)-1-(4-Ethylphenyl)-2-methyl-3-piperidin-1-ylpropan-1-one according to claim 2.

9. A pharmaceutical composition comprising the substantially enantiopure crystalline maleate salt of (2RS)-1-(4-Ethylphenyl)-2-methyl-3-piperidin-1-ylpropan-1-one according to claim 3.

10-24. (canceled)

25. A method of treating and/or preventing any of the following conditions, comprising administering a pharmaceutical composition comprising a substantially enantiopure crystalline salt of (2RS)-1-(4-Ethylphenyl)-2-methyl-3-piperidin-1-ylpropan-1-one, wherein said substantially enantiopure crystalline salt of (2RS)-1-(4-Ethylphenyl)-2-methyl-3-piperidin-1-ylpropan-1-one is chosen from a substantially enantiopure crystalline hydrochloride salt, a substantially enantiopure crystalline mesylate salt, and a substantially enantiopure crystalline maleate salt: discomfort, muscle spasm, stiffness, or myotonic conditions associated with musculoskeletal conditions; spasticity or spastic paralysis of neurological origin; dystonia; headache; fibromyalgia; chronic fatigue syndrome; muscle cramps; pain of various etiologies; and disorders that arise from altered cell membrane excitability.
